SEQUENCE LISTING (7 sequences)

SEQ ID NO: 1 - protein, 381 aa, Homo sapiens
MSGVVRTLSRCLLPAEAGGARERRAGSGARDAEREARRRSRDIDALLARERRAVRRLVKILLLGAGESGKSTFLKQMRIIHGREFDQKALLEFRDTIFDNILKGSRVLVDARDKLGIPWQYSENEKHGMFLMAFENKAGLPVEPATFQLYVPALSALWRDSGIREAFSRRSEFQLGESVKYFLDNLDRIGQLNYFPSKQDILLARKATKGIVEHDFVIKKIPFKMVDVGGQRSQRQKWFQCFDGITSILFMVSSSEYDQVLMEDRRTNRLVESMNIFETIVNNKLFFNVSIILFLNKMDLLVEKVKTVSIKKHFPDFRGDPHRLEDVQRYLVQCFDRKRRNRSKPLFHHFTTAIDTENVRFVFHAVKDTILQENLKDIMLQ

SEQ ID NO: 2 - protein, 1044 aa, Homo sapiens
MPPGVDCPMEFWTKEENQSVVVDFLLPTGVYLNFPVSRNANLSTIKQLLWHRAQYEPLFHMLSGPEAYVFTCINQTAEQQELEDEQRRLCDVQPFLPVLRLVAREGDRVKKLINSQISLLIGKGLHEFDSLCDPEVNDFRAKMCQFCEEAAARRQQLGWEAWLQYSFPLQLEPSAQTWGPGTLRLPNRALLVNVKFEGSEESFTFQVSTKDVPLALMACALRKKATVFRQPLVEQPEDYTLQVNGRHEYLYGSYPLCQFQYICSCLHSGLTPHLTMVHSSSILAMRDEQSNPAPQVQKPRAKPPPIPAKKPSSVSLWSLEQPFRIELIQGSKVNADERMKLVVQAGLFHGNEMLCKTVSSSEVSVCSEPVWKQRLEFDINICDLPRMARLCFALYAVIEKAKKARSTKKKSKKADCPIAWANLMLFDYKDQLKTGERCLYMWPSVPDEKGELLNPTGTVRSNPNTDSAAALLICLPEVAPHPVYYPALEKILELGRHSECVHVTEEEQLQLREILERRGSGELYEHEKDLVWKLRHEVQEHFPEALARLLLVTKWNKHEDVAQMLYLLCSWPELPVLSALELLDFSFPDCHVGSFAIKSLRKLTDDELFQYLLQLVQVLKYESYLDCELTKFLLDRALANRKIGHFLFWHLRSEMHVPSVALRFGLILEAYCRGSTHHMKVLMKQGEALSKLKALNDFVKLSSQKTPKPQTKELMHLCMRQEAYLEALSHLQSPLDPSTLLAEVCVEQCTFMDSKMKPLWIMYSNEEAGSGGSVGIIFKNGDDLRQDMLTLQMIQLMDVLWKQEGLDLRMTPYGCLPTGDRTGLIEVVLRSDTIANIQLNKSNMAATAAFNKDALLNWLKSKNPGEALDRAIEEFTLSCAGYCVATYVLGIGDRHSDNIMIRESGQLFHIDFGHFLGNFKTKFGINRERVPFILTYDFVHVIQQGKTNNSEKFERFRGYCERAYTILRRHGLLFLHLFALMRAAGLPELSCSKDIQYLKDSLALGKTEEEALKHFRVKFNEALRESWKTKVNWLAHNVSKDNRQ

SEQ ID NO: 3 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 4 - protein, 912 aa, Homo sapiens
MEDFARGAASPGPSRPGLVPVSIIGAEDEDFENELETNSEEQNSQFQSLEQVKRRPAHLMALLQHVALQFEPGPLLCCLHADMLGSLGPKEAKKAFLDFYHSFLEKTAVLRVPVPPNVAFELDRTRADLISEDVQRRFVQEVVQSQQVAVGRQLEDFRSKRLMGMTPWEQELAQLEAWVGRDRASYEARERHVAERLLMHLEEMQHTISTDEEKSAAVVNAIGLYMRHLGVRTKSGDKKSGRNFFRKKVMGNRRSDEPAKTKKGLSSILDAARWNRGEPQVPDFRHLKAEVDAEKPGATDRKGGVGMPSRDRNIGAPGQDTPGVSLHPLSLDSPDREPGADAPLELGDSSPQGPMSLESLAPPESTDEGAETESPEPGDEGEPGRSGLELEPEEPPGWRELVPPDTLHSLPKSQVKRQEVISELLVTEAAHVRMLRVLHDLFFQPMAECLFFPLEELQNIFPSLDELIEVHSLFLDRLMKRRQESGYLIEEIGDVLLARFDGAEGSWFQKISSRFCSRQSFALEQLKAKQRKDPRFCAFVQEAESRPRCRRLQLKDMIPTEMQRLTKYPLLLQSIGQNTEEPTEREKVELAAECCREILHHVNQAVRDMEDLLRLKDYQRRLDLSHLRQSSDPMLSEFKNLDITKKKLVHEGPLTWRVTKDKAVEVHVLLLDDLLLLLQRQDERLLLKSHSRTLTPTPDGKTMLRPVLRLTSAMTREVATDHKAFYVLFTWDQEAQIYELVAQTVSERKNWCALITETAGSLKVPAPASRPKPRPSPSSTREPLLSSSENGNGGRETSPADARTERILSDLLPFCRPGPEGQLAATALRKVLSLKQLLFPAEEDNGAGPPRDGDGVPGGGPLSPARTQEIQENLLSLEETMKQLEELEEEFCRLRPLLSQLGGNSVPQPGCT

SEQ ID NO: 5 - protein, 1354 aa, Homo sapiens
MSTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNRTSSNADKSLQESLQKTIYKLEEQLHNEMQLKDEMEQKCRTSNIKLDKIMKELDEEGNQRRNLESTVSQIEKEKMLLQHRINEYQRKAEQENEKRRNVENEVSTLKDQLEDLKKVSQNSQLANEKLSQLQKQLEEANDLLRTESDTAVRLRKSHTEMSKSISQLESLNRELQERNRILENSKSQTDKDYYQLQAILEAERRDRGHDSEMIGDLQARITSLQEEVKHLKHNLEKVEGERKEAQDMLNHSEKEKNNLEIDLNYKLKSLQQRLEQEVNEHKVTKARLTDKHQSIEEAKSVAMCEMEKKLKEEREAREKAENRVVQIEKQCSMLDVDLKQSQQKLEHLTGNKERMEDEVKNLTLQLEQESNKRLLLQNELKTQAFEADNLKGLEKQMKQEINTLLEAKRLLEFELAQLTKQYRGNEGQMRELQDQLEAEQYFSTLYKTQVKELKEEIEEKNRENLKKIQELQNEKETLATQLDLAETKAESEQLARGLLEEQYFELTQESKKAASRNRQEITDKDHTVSRLEEANSMLTKDIEILRRENEELTEKMKKAEEEYKLEKEEEISNLKAAFEKNINTERTLKTQAVNKLAEIMNRKDFKIDRKKANTQDLRKKEKENRKLQLELNQEREKFNQMVVKHQKELNDMQAQLVEECAHRNELQMQLASKESDIEQLRAKLLDLSDSTSVASFPSADETDGNLPESRIEGWLSVPNRGNIKRYGWKKQYVVVSSKKILFYNDEQDKEQSNPSMVLDIDKLFHVRPVTQGDVYRAETEEIPKIFQILYANEGECRKDVEMEPVQQAEKTNFQNHKGHEFIPTLYHFPANCDACAKPLWHVFKPPPALECRRCHVKCHRDHLDKKEDLICPCKVSYDVTSARDMLLLACSQDEQKKWVTHLVKKIPKNPPSGFVRASPRTLSTRSTANQSFRKVVKNTSGKTS

SEQ ID NO: 6 - protein, 1388 aa, Homo sapiens
MSRPPPTGKMPGAPETAPGDGAGASRQRKLEALIRDPRSPINVESLLDGLNSLVLDLDFPALRKNKNIDNFLNRYEKIVKKIRGLQMKAEDYDVVKVIGRGAFGEVQLVRHKASQKVYAMKLLSKFEMIKRSDSAFFWEERDIMAFANSPWVVQLFYAFQDDRYLYMVMEYMPGGDLVNLMSNYDVPEKWAKFYTAEVVLALDAIHSMGLIHRDVKPDNMLLDKHGHLKLADFGTCMKMDETGMVHCDTAVGTPDYISPEVLKSQGGDGFYGRECDWWSVGVFLYEMLVGDTPFYADSLVGTYSKIMDHKNSLCFPEDAEISKHAKNLICAFLTDREVRLGRNGVEEIRQHPFFKNDQWHWDNIRETAAPVVPELSSDIDSSNFDDIEDDKGDVETFPIPKAFVGNQLPFIGFTYYRENLLLSDSPSCRETDSIQSRKNEESQEIQKKLYTLEEHLSNEMQAKEELEQKCKSVNTRLEKTAKELEEEITLRKSVESALRQLEREKALLQHKNAEYQRKADHEADKKRNLENDVNSLKDQLEDLKKRNQNSQISTEKVNQLQRQLDETNALLRTESDTAARLRKTQAESSKQIQQLESNNRDLQDKNCLLETAKLKLEKEFINLQSALESERRDRTHGSEIINDLQGRICGLEEDLKNGKILLAKVELEKRQLQERFTDLEKEKSNMEIDMTYQLKVIQQSLEQEEAEHKATKARLADKNKIYESIEEAKSEAMKEMEKKLLEERTLKQKVENLLLEAEKRCSLLDCDLKQSQQKINELLKQKDVLNEDVRNLTLKIEQETQKRCLTQNDLKMQTQQVNTLKMSEKQLKQENNHLMEMKMNLEKQNAELRKERQDADGQMKELQDQLEAEQYFSTLYKTQVRELKEECEEKTKLGKELQQKKQELQDERDSLAAQLEITLTKADSEQLARSIAEEQYSDLEKEKIMKELEIKEMMARHKQELTEKDATIASLEETNRTLTSDVANLANEKEELNNKLKDVQEQLSRLKDEEISAAAIKAQFEKQLLTERTLKTQAVNKLAEIMNRKEPVKRGNDTDVRRKEKENRKLHMELKSEREKLTQQMIKYQKELNEMQAQIAEESQIRIELQMTLDSKDSDIEQLRSQLQALHIGLDSSSIGSGPGDAEADDGFPESRLEGWLSLPVRNNTKKFGWVKKYVIVSSKKILFYDSEQDKEQSNPYMVLDIDKLFHVRPVTQTDVYRADAKEIPRIFQILYANEGESKKEQEFPVEPVGEKSNYICHKGHEFIPTLYHFPTNCEACMKPLWHMFKPPPALECRRCHIKCHKDHMDKKEEIIAPCKVYYDISTAKNLLLLANSTEEQQKWVSRLVKKIPKKPPAPDPFARSSPRTSMKIQQNQSIRRPSRQLAPNKPS

SEQ ID NO: 7 - protein, 135 aa, Homo sapiens
MELGAKIRLNSVLYAMNNTEESCLPQLFEIFHEKIDFDQCIRPILFGEITYSLTENHDCILSYGANINYLDENGNSPLIVAAENGLKSLIRYLIRNGADASIKNHQGKTAFDVCDKSIQTYMRKMMKKYNRVQTH

(19) United States
(12) Patent Application Publication
(10) Pub. No.: US 2025/0057836 A1
(43) Pub. Date: Feb. 20, 2025

(54) Compositions And Methods Targeting G12 Signaling For Bronchodilator Therapy

(71) Applicant: Rutgers, The State University of New Jersey, New Brunswick, NJ (US)

(72) Inventors: Reynold Panettieri, New Brunswick, NJ (US); Edwin Yoo, New Brunswick, NJ (US)

(21) Appl. No.: 18/758,924

(22) Filed: Jun. 28, 2024

Related U.S. Application Data
(63) Continuation of application No. 16/622,813, filed on Dec. 13, 2019, now abandoned.
(60) Provisional application No. 62/526,727, filed on Jun. 29, 2017.

(51) Int. Cl. A61K 31/498; A61K 31/137; A61K 38/16; A61K 45/06; C12N 15/113; C12Q 1/50; C12Q 1/66; G01N 33/50

(52) U.S. Cl. CPC A61K 31/498; A61K 31/137; A61K 38/16; A61K 38/164; A61K 45/06; C12N 15/113; C12Q 1/50; C12Q 1/66; G01N 33/5041; C12N 2310/141

(57) ABSTRACT

Provided herein are methods for inhibiting contraction and/or promoting relaxation of airway smooth muscle cells (e.g., human airway smooth muscle cells), comprising contacting the cells with a Gα₁₂ or RhoA inhibitor. Also provided herein are methods for inhibiting and/or treating bronchoconstriction or promoting bronchodilation in a subject, for example, a subject with airway hyperresponsiveness and/or a disease associated with bronchoconstriction, such as asthma, chronic obstructive pulmonary disease, chronic bronchitis, bronchiectasis or cystic fibrosis, using a Gα₁₂ or RhoA inhibitor, as well as pharmaceutical compositions comprising a Gα₁₂ or RhoA inhibitor.

### RELATED APPLICATION(S)

This application is a Continuation of U.S. application Ser. No. 16/622,813, which is the U.S. National Stage of International Application No. PCT/US2018/037773, filed on Jun. 15, 2018, published in English, which claims the benefit of U.S. Provisional Application No. 62/526,727, filed on Jun. 29, 2017. The entire teachings of the above applications are incorporated herein by reference.

### GOVERNMENT SUPPORT

This invention was made with government support under grant number HL114471 awarded by the National Institutes of Health. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith:
a) File name: 5431.1001-003 Sequence Listing.xml; created Jun. 11, 2024, 12,268 Bytes in size.

### BACKGROUND

Airway Hyperresponsiveness (AHR), a hallmark of asthma, represents exaggerated airway narrowing in response to contractile agonists such as acetylcholine (Koziol-White and Panettieri, 2011; Panettieri, 2016). Human airway smooth muscle cells (HASMCs) mediate AHR by shortening in response to contractile agonists (Amrani et al., 2004). Inhibition of calcium sensitization pathways in human airway smooth muscle has been shown to be an effective method of inducing bronchodilation. Due to off target effects in vascular smooth muscle, however, clinical trials of bronchodilators targeting calcium sensitization pathways have been precluded.

Accordingly, there is a need for alternative approaches to the clinical management of airway obstruction in asthma, chronic obstructive pulmonary disease, cystic fibrosis and other inflammatory lung diseases.

### SUMMARY

Provided herein are methods for inhibiting contraction of an ASMC (to inhibit or treat bronchoconstriction, for example), and methods for promoting relaxation of an ASMC (to promote bronchodilation or treat bronchoconstriction, for example). The invention described herein is based, at least in part, on the discovery that Gα₁₂ plays an important role in HASMC contraction via RhoA-dependent activation of the PI3K/ROCK axis.

Accordingly, provided herein is a method of inhibiting contraction of an airway smooth muscle cell (ASMC), the method comprising contacting the ASMC with a Gα₁₂ inhibitor.

Also provided is a method of promoting relaxation of an ASMC, the method comprising contacting the ASMC with a Gα₁₂ inhibitor.

Also provided is a method of inhibiting bronchoconstriction in a subject in need thereof, comprising administering to the subject a therapeutically effective amount of a Gα₁₂ inhibitor.

Also provided is a method of promoting bronchodilation in a subject in need thereof, comprising administering to the subject a therapeutically effective amount of a Gα₁₂ inhibitor.

Also provided is a method of treating bronchoconstriction in a subject in need thereof, comprising administering to the subject a therapeutically effective amount of a Gα₁₂ inhibitor.

Also provided is a pharmaceutical composition comprising a Gα₁₂ inhibitor and a pharmaceutically acceptable carrier.

Also provided herein is a method of inhibiting contraction of an ASMC, the method comprising contacting the ASMC with a ras homolog gene family, member A (RhoA) inhibitor.

Also provided is a method of promoting relaxation of an ASMC, the method comprising contacting the ASMC with a RhoA inhibitor.

Also provided is a method of inhibiting bronchoconstriction in a subject in need thereof, comprising administering to the subject a therapeutically effective amount of a RhoA inhibitor.

Also provided is a method of promoting bronchodilation in a subject in need thereof, comprising administering to the subject a therapeutically effective amount of a RhoA inhibitor.

Also provided is a method of treating bronchoconstriction in a subject in need thereof, comprising administering to the subject a therapeutically effective amount of a RhoA inhibitor.

Also provided is a pharmaceutical composition comprising a RhoA inhibitor and a pharmaceutically acceptable carrier.

Also provided is a method of identifying an agent that inhibits contraction or promotes relaxation of an ASMC. The method comprises contacting an ASMC with a contractile agent and a candidate agent that inhibits contraction or promotes relaxation of an ASMC, and measuring activation of the PI3K/ROCK axis in the ASMC. A reduction in activation of the PI3K/ROCK axis in an ASMC that has been contacted with the candidate agent compared to a control indicates that the candidate agent inhibits contraction or promotes relaxation of an ASMC.

Inhibiting Gα₁₂ signaling in human airway smooth muscle promotes bronchodilation by blocking calcium sensitization pathways, and may be more effective and tissue-specific than traditional approaches to inhibiting calcium sensitization pathways. Accordingly, the inventions described herein represent alternative approaches to the clinical management of airway obstruction in asthma, chronic obstructive pulmonary disease, cystic fibrosis and other inflammatory lung diseases.

### BRIEF DESCRIPTION OF THE DRAWINGS

The foregoing will be apparent from the following more particular description of example embodiments.

FIGS. 1A-1E show the effects of M2R siRNA, M3R siRNA, and pertussis toxin on carbachol-induced AKT and MLC phosphorylation in primary HASMCs. (A-C) Measurement of phosphorylation responses to carbachol (10 μM, 10 minutes) and protein expression in primary HASMCs after transfection with M2R, M3R, or scrambled siRNA (50 nM, 72 hours post-transfection). (A) Effect of scrambled, M2R and M3R siRNA on protein expression. Data normalized to GAPDH expression in the same samples. (B) Effect of carbachol on AKT phosphorylation at S473 (pAKT) after transfection with scrambled, M2R, or M3R siRNA. pAKT data were normalized to total AKT (AKT). (C) Effect of carbachol on MLC phosphorylation at S19 (pMLC) after transfection with scrambled, M2R, or M3R siRNA. pMLC data were normalized to total MLC (MLC). (D) Effect of pertussis toxin (18 hours, 1 μg ml⁻¹) on carbachol-induced AKT phosphorylation. Data normalized to tubulin expression in the same samples. (E) Effect of pertussis toxin (18 hours, 1 μg ml⁻¹) on carbachol-induced attenuation of isoproterenol-mediated cAMP production. Data are expressed as fold change over untreated (basal) samples that were measured on the same gel or plate. Data are representative of five independent experiments (n=5, mean±SD); statistical comparisons analyzed by one-way ANOVA with Bonferroni post-test and significant comparisons are denoted by lines between tested conditions. *P<0.05.

FIGS. 2A and 2B show Gα₁₂ and M3R coupling in HASMCs. Evaluation of M3R-Gα₁₂ coupling using co-immunoprecipitation in primary HASMCs and SRE-luciferase reporter in hTERT-immortalized HASMCs expressing p115RhoGEF-RGS. (A) HASMCs were stimulated with carbachol (10 μM, 1 minute) and lysates were immunoprecipitated with anti-M3R or anti-Gα₁₂ antibody and then probed as indicated. Immunoblot is representative of five independent experiments. (B) hTERT-immortalized HASMCs expressing p115RhoGEF-RGS and control hTERT-immortalized HASMCs (post G418 selection) were infected with SRE-luciferase reporter. After carbachol stimulation (10 μM, 6 hours), cells were lysed and SRE-luciferase reporter activity was measured. Data are expressed as fold change over untreated (basal) samples that were measured on the same plate. Data are representative of six independent experiments (n=6, mean±SD); statistical comparisons analyzed by one-way ANOVA with Bonferroni post-test and significant comparisons are denoted by lines between tested conditions. *P<0.05.

FIGS. 3A-3G show the effects of Gα₁₂ siRNA and p115RhoGEF-RGS overexpression on M3R-mediated activation of the PI3K/ROCK/MLC axis in HASMCs. (A-C) Measurement of phosphorylation responses to carbachol (10 μM, 10 minutes) and protein expression in primary HASMCs after transfection with Gα₁₂ or scrambled siRNA (50 nM, 72 hours post-transfection). (A) Effect of Gα₁₂ or scrambled siRNA on protein expression. Data normalized to tubulin expression in the same samples. (B) Effect of carbachol on AKT, MYPT1, and MLC phosphorylation at S473 (pAKT), T696 (pMYPT1), and S19 (pMLC) after transfection with Gα₁₂ or scrambled siRNA. pAKT, pMYPT1, and pMLC data were normalized to total AKT (AKT), total MYPT1 (MYPT1), and total MLC (MLC). (C) Effect of p115RhoGEF-RGS overexpression on carbachol-induced AKT phosphorylation in hTERT-immortalized HASMCs. Control refers to hTERT-immortalized HASMCs that underwent G418 selection. (D) Effect of p115RhoGEF-RGS overexpression on carbachol-induced intracellular calcium mobilization in hTERT-immortalized HASMCs. Data are expressed as fold change over untreated (basal) samples that were measured on the same gel or plate. Data are representative of five independent donors (n=5, mean±SD). (E) Effect of p115RhoGEF-RGS overexpression on carbachol-induced contraction in hTERT-immortalized HASMCs as measured by MTC analysis of isolated airway smooth muscle (ASM) (control, n=278; p115RhoGEF-RGS, n=237). (F) Representative images of a typical modified HASM cell responding to carbachol. A single nucleus confirms the presence of one cell. The addition of carbachol induces increased force generation by the cell onto the contractible fluorescent micropattern, resulting in a smaller pattern over time. (G) Quantification of cell contraction to carbachol in p115RhoGEF-RGS-expressing and control HASMCs. Line plots depict the evolution of contractile forces, shown as the median population-wide responsiveness in each of 36 technical experimental replicates (thin gray lines) and their mean (heavy lines) for both the p115RhoGEF-RGS-expressing and control HASMCs. Comparison of the heavy lines demonstrates a significant inhibition in contractile responsiveness in p115RhoGEF-RGS-expressing and control HASMCs. Bars represent SEM, with each thin gray line representing between 13-52 isolated cells analyzed per replicate, corresponding to >800 total cells analyzed per condition. Data are representative of five biological replicates (n=5, mean±SEM); statistical comparisons analyzed by unpaired t-test are denoted by asterisks. *P<0.05. Statistical comparisons analyzed by one-way ANOVA with Bonferroni post-test are denoted by lines between tested conditions. Control in all experiments refers to hTERT-immortalized HASMCs that underwent G418 selection.

FIGS. 4A-4C show the effects of RhoA inhibitors and siRNA on M3R-mediated activation of PI3K in primary HASMCs. Measurement of phosphorylation responses to carbachol (10 μM, 10 minutes) and protein expression in primary HASMCs after transfection with RhoA, Rac1, or scrambled siRNA (50 nM, 72 hours post-transfection) or after incubation with rhosin (RhoA inhibitor) (10 μM, 30 minutes). (A) Effect of scrambled, RhoA and Rac1 siRNA on protein expression. Data normalized to MLC expression in the same samples. (B) Effect of carbachol on AKT phosphorylation at S473 (pAKT) after transfection with RhoA, Rac1, or scrambled siRNA. pAKT data were normalized to total AKT (AKT). (C) Effect of rhosin on carbachol-induced AKT phosphorylation at S473 (pAKT). Data are expressed as fold change over untreated (basal) samples that were measured on the same gel. Data are representative of five independent experiments (n=5, mean±SD); statistical comparisons analyzed by one-way ANOVA with Bonferroni post-test and significant comparisons are denoted by lines between tested conditions. *P<0.05.

FIG. 5 shows that RhoA inhibition reverses carbachol-induced bronchoconstriction in a dose-dependent manner in human precision-cut lung slices (hPCLS). Measurement of bronchodilation concentration-responses to rhosin in hPCLS. Airways were preconstricted to carbachol (10⁻⁸-10⁻⁴ M) prior to dilation to rhosin or formoterol (10⁻¹⁰-10⁻⁴ M). Data were normalized to forskolin stimulation (10 μM) that was given after the final dose of formoterol or rhosin. Each data point is expressed as mean±SEM. Each group contains 2 airways from each of three donors (6 total airways).

### DETAILED DESCRIPTION

A description of example embodiments follows.

Activation of the phosphoinositide 3-kinase/rho kinase (PI3K/ROCK) axis is necessary for agonist-induced human airway smooth muscle cell (HASMC) contraction, and inhibition of the PI3K/ROCK axis promotes bronchodilation of human small airways. The PI3K/ROCK axis includes the following proteins: Gα₁₂; phosphoinositide 3-kinase (PI3K) delta; ras homolog gene family, member A (RhoA); rho guanine nucleotide exchange factor (RhoGEF); rho kinase (ROCK); and myosin light chain phosphatase (MLCP).

Acetylcholine release from postganglionic parasympathetic nerves innervating the airway activates the M3-muscarinic acetylcholine receptor (M3R), a G protein-coupled receptor expressed by HASMCs (Billington and Penn, 2002). Stimulation of the M3R evokes Gα_{q/11-}mediated calcium release from the sarcoplasmic reticulum, resulting in MLC kinase (MLCK) activation and myosin light chain (MLC) phosphorylation. MLC phosphorylation induces actomyosin cross-bridge cycling and HASMC shortening (Billington and Penn, 2003). In parallel, activation of Rho kinase (ROCK) by the small GTPase RhoA, phosphorylates and inactivates MLC phosphatase (MLCP). Inhibition of the constitutively active MLCP augments and sustains MLC phosphorylation and maintenance of HASMC contraction (Chiba and Misawa, 2004; Chiba et al., 2010).

Phosphoinositide 3-kinase (PI3K), a lipid kinase, is a necessary mediator of muscarinic receptor-induced ROCK activation and human airway bronchoconstriction, and PI3K inhibitors can reverse carbachol-induced bronchoconstriction by attenuating PI3K/ROCK-axis activation (Koziol-White et al., 2016). The therapeutic importance of ROCK signaling is emphasized by the ability of ROCK and PI3K inhibitors to promote bronchodilation. However, the upstream mechanisms regulating muscarinic receptor-induced ROCK and PI3K activation in HASMC remain unclear (Pera and Penn, 2016).

Gα_{12/13} family members, including Gα₁₂ and Gα₁₃, promote ROCK signaling by activating Rho guanine nucleotide exchange factors (RhoGEFs), including p115RhoGEF, which exchange GDP for GTP and activate RhoA (Siehler, 2009). p115RhoGEF contains a regulator of G-protein signaling (RGS) domain, that specifically limits Gα_{12/13} signaling after activation (Wells et al., 2002). Gα_{12/13} proteins mediate various cell functions including stress fiber formation, cytoskeletal rearrangement, and proliferation (Riobo and Manning, 2005; Worzfeld et al., 2008). In the context of HASMC function, however, Gα_{12/13} signaling remains poorly understood.

As used herein, “Gα₁₂” refers to a protein having the amino acid sequence of human Gα₁₂ assigned National Center for Biotechnology Information (NCBI) Accession No. NP_001269370 (SEQ ID NO:1), or a variant thereof having at least about 70% (e.g., about 75%, about 80%, about 85%, about 90%, about 95%, about 96%, about 97%, about 98% or about 99%) identity to the amino acid sequence of SEQ ID NO:1. “Gα₁₂” includes naturally occurring or endogenous Gα₁₂ proteins (e.g., a mammalian, in particular, a human, Gα₁₂ protein), and proteins having an amino acid sequence that is the same as that of a naturally occurring or endogenous Gα₁₂ protein (e.g., a recombinant or synthetic protein). Accordingly, “Ga₁₂” includes naturally occurring variants and other isoforms of a Gα₁₂ protein produced by, e.g., alternative splicing or other cellular processes that occur naturally in mammals (e.g., humans). In some embodiments, the Gα₁₂ protein has the amino acid sequence of SEQ ID NO:1.

As used herein, the term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. The sequence identity comparison can be examined throughout the entire length of a given protein, or within a desired fragment of a given protein. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

As used herein, “phosphoinositide 3-kinase” and “PI3K” refer to a protein having the amino acid sequence of human PI3K assigned NCBI Accession No. NP_005017 (SEQ ID NO:2), or a variant thereof having at least about 70% (e.g., about 75%, about 80%, about 85%, about 90%, about 95%, about 96%, about 97%, about 98% or about 99%) identity to the amino acid sequence of SEQ ID NO:2. “Phosphoinositide 3-kinase” and “PI3K” include naturally occurring or endogenous PI3K proteins (e.g., a mammalian, in particular, a human, PI3K protein), and proteins having an amino acid sequence that is the same as that of a naturally occurring or endogenous PI3K protein (e.g., a recombinant or synthetic protein). Accordingly, “phosphoinositide 3-kinase” and “PI3K” include naturally occurring variants and other isoforms of a PI3K protein produced by, e.g., alternative splicing or other cellular processes that occur naturally in mammals (e.g., humans). In some embodiments, the PI3K protein has the amino acid sequence of SEQ ID NO:2.

As used herein, “Ras homolog gene family, member A” and “RhoA” refer to a protein having the amino acid sequence of human RhoA assigned NCBI Accession No. NP_001300870 (SEQ ID NO:3), or a variant thereof having at least about 70% (e.g., about 75%, about 80%, about 85%, about 90%, about 95%, about 96%, about 97%, about 98% or about 99%) identity to the amino acid sequence of SEQ ID NO:3. “Ras homolog gene family, member A” and “RhoA” include naturally occurring or endogenous RhoA proteins (e.g., a mammalian, in particular, a human, RhoA protein), and proteins having an amino acid sequence that is the same as that of a naturally occurring or endogenous RhoA protein (e.g., a recombinant or synthetic protein). Accordingly, “Ras homolog gene family, member A” and “RhoA” include naturally occurring variants and other isoforms of a RhoA protein produced by, e.g., alternative splicing or other cellular processes that occur naturally in mammals (e.g., humans). In some embodiments, the RhoA protein has the amino acid sequence of SEQ ID NO:3.

As used herein, “rho guanine nucleotide exchange factor” and “RhoGEF” refer to a protein having the amino acid sequence of human RhoGEF assigned UniProt Accession No. Q92888 (SEQ ID NO:4), or a variant thereof having at least about 70% (e.g., about 75%, about 80%, about 85%, about 90%, about 95%, about 96%, about 97%, about 98% or about 99%) identity to the amino acid sequence of SEQ ID NO:5. “Rho guanine nucleotide exchange factor” and “RhoGEF” include naturally occurring or endogenous RhoGEF proteins (e.g., a mammalian, in particular, a human, RhoGEF protein), and proteins having an amino acid sequence that is the same as that of a naturally occurring or endogenous RhoGEF protein (e.g., a recombinant or synthetic protein). Accordingly, “rho guanine nucleotide exchange factor” and “RhoGEF” include naturally occurring variants and other isoforms of a RhoGEF protein produced by, e.g., alternative splicing or other cellular processes that occur naturally in mammals (e.g., humans). In some embodiments, the RhoGEF protein has the amino acid sequence of SEQ ID NO:4.

As used herein, “rho kinase” and “ROCK” refer to a protein having the amino acid sequence of human ROCK1 assigned UniProt Accession No. Q13464 (SEQ ID NO:5) or the amino acid sequence of human ROCK2 assigned UniProt Accession No. 075116 (SEQ ID NO:6), or a variant of any of the foregoing having at least about 70% (e.g., about 75%, about 80%, about 85%, about 90%, about 95%, about 96%, about 97%, about 98% or about 99%) identity to the amino acid sequence of SEQ ID NO:5 or SEQ ID NO:6. “Rho kinase” and “ROCK” include naturally occurring or endogenous ROCK proteins (e.g., a mammalian, in particular, a human, ROCK protein), and proteins having an amino acid sequence that is the same as that of a naturally occurring or endogenous ROCK protein (e.g., a recombinant or synthetic protein). Accordingly, “rho kinase” and “ROCK” include naturally occurring variants and other isoforms of a ROCK protein produced by, e.g., alternative splicing or other cellular processes that occur naturally in mammals (e.g., humans). In some embodiments, the ROCK protein has the amino acid sequence of SEQ ID NO:5. In some embodiments, the ROCK protein has the amino acid sequence of SEQ ID NO:6.

As used herein, “myosin light chain phosphatase” and “MLCP” refer to a protein having the amino acid sequence of human MLCP assigned UniProt Accession No. A2D9C4 (SEQ ID NO:7), or a variant thereof having at least about 70% (e.g., about 75%, about 80%, about 85%, about 90%, about 95%, about 96%, about 97%, about 98% or about 99%) identity to the amino acid sequence of SEQ ID NO:7. “Myosin light chain phosphatase” and “MLCP” include naturally occurring or endogenous MLCP proteins (e.g., a mammalian, in particular, a human, MLCP protein), and proteins having an amino acid sequence that is the same as that of a naturally occurring or endogenous MLCP protein (e.g., a recombinant or synthetic protein). Accordingly, “myosin light chain phosphatase” and “LCP” include naturally occurring variants and other isoforms of a MLCP protein produced by, e.g., alternative splicing or other cellular processes that occur naturally in mammals (e.g., humans). In some embodiments, the MLCP protein has the amino acid sequence of SEQ ID NO:7.

### Methods of Treatment

Provided herein is a method of inhibiting contraction of an airway smooth muscle cell (ASMC) (e.g., a human ASMC (HASMC)), the method comprising contacting the ASMC with a Gα₁₂ inhibitor or a RhoA inhibitor (e.g., an effective amount of a Gα₁₂ inhibitor or a RhoA inhibitor). In some embodiments, the method comprises contacting the ASMC (e.g., HASMC) with a Gα₁₂ inhibitor (e.g., an effective amount of a Gα₁₂ inhibitor). In some embodiments, the method comprises contacting the ASMC (e.g., HASMC) with a RhoA inhibitor (e.g., an effective amount of a RhoA inhibitor). In some embodiments, the method comprises contacting the ASMC (e.g., HASMC) with a Gα₁₂ inhibitor (e.g., an effective amount of a Gα₁₂ inhibitor) and a RhoA inhibitor (e.g., an effective amount of a RhoA inhibitor).

As used herein, the singular forms “a”, “an” and “the” include plural referents unless the context clearly dictates otherwise. Thus, for example, reference to “a Gα₁₂ inhibitor” can include a plurality of Gα₁₂ inhibitors. Further, the plurality can comprise more than one of the same Gα₁₂ inhibitors or a plurality of different Gα₁₂ inhibitors.

As used herein, “inhibits” means reduces, decreases or prevents, either partially or entirely.

“Contraction,” as used herein with respect to a cell, refers to a shortening in length or increase in tension of a cell, such as an ASMC (e.g., HASMC). An increase in stiffness of a cell, as compared to the stiffness of the cell in its relaxed, natural or low-tension state, for example, is considered an index of single-cell smooth muscle contraction. Stiffness can be measured by magnetic twisting cytometry, as described herein.

As used herein, an “effective amount” is an amount sufficient to achieve a desired effect under the conditions of administration, in vitro, in vivo or ex vivo, such as, for example, an amount sufficient to inhibit contraction of a cell (e.g., an ASMC, such as a HASMC) or an amount sufficient to promote relaxation of a cell (e.g., an ASMC, such as a HASMC), for example, in a subject. The effectiveness of a therapy can be determined by suitable methods known by those of skill in the art including those described herein.

“Gα₁₂ inhibitor,” as used herein, refers to any agent that inhibits the signaling activity of Gα₁₂, either directly (e.g., as a Gα₁₂ inverse agonist or antagonist) or indirectly (e.g., by inhibiting formation of the Gα₁₂-M3 muscarinic acetylcholine receptor (M3R) complex or upregulating p115RhoGEF, and thereby disrupting Gα₁₂ signaling). In some embodiments, the Gα₁₂ inhibitor is a direct inhibitor, preferably, a Gα₁₂ antagonist. In other embodiments, the Gα₁₂ inhibitor is an indirect inhibitor.

Non-limiting examples of Gα₁₂ inhibitors include a nucleic acid (e.g., a short interfering ribonucleic acid (siRNA)), a peptide (e.g., a polypeptide comprising a regulator of G-protein signaling (RGS) domain), an antibody, a peptidomimetic or a small molecule.

“Ras homolog gene family, member A inhibitor” and “RhoA inhibitor,” as used herein, refer to any agent that inhibits the signaling activity of RhoA, either directly (e.g., as a RhoA inverse agonist or antagonist) or indirectly. In some embodiments, the RhoA inhibitor is a direct inhibitor, preferably, a RhoA antagonist, such as rhosin. In other embodiments, the RhoA inhibitor is an indirect inhibitor.

Non-limiting examples of RhoA inhibitors include a nucleic acid (e.g., a short interfering ribonucleic acid (siRNA)), a peptide, an antibody, a peptidomimetic or a small molecule (e.g., rhosin).

As used herein, the term “nucleic acid” refers to a compound consisting of two or more nucleotides, each nucleotide being made of a five-carbon sugar, a phosphate group and a nitrogenous base. Nucleic acid inhibitors useful in the present invention include deoxyribonucleic acid (DNA) and ribonucleic acid (RNA), for example, siRNA. Nucleic acid inhibitors also include aptamers, which are capable of binding to a particular molecule of interest (e.g., Gα₁₂, RhoA) with high affinity and specificity through interactions other than classic Watson-Crick base pairing (Tuerk and Gold, Science 249:505 (1990); Ellington and Szostak, Nature 346:818 (1990)). A typical aptamer is 10-15 kDa in size (30-45 nucleotides), binds its target with sub-nanomolar affinity, and discriminates against closely related targets (e.g., will typically not bind other proteins from the same gene family). Aptamers can be generated and identified using a standard process known as “Systematic Evolution of Ligands by Exponential Enrichment” (SELEX), described in, e.g., U.S. Pat. Nos. 5,475,096 and 5,270,163.

As used herein, the term “peptide” refers to a compound consisting of two or more linked amino acids, wherein the amino group of one amino acid is joined to the carboxyl group of another amino acid by an amide bond. Peptides are typically less than about 100 amino acid residues in length and preferably are about 10, about 20, about 30, about 40 or about 50 amino acid residues in length. In one embodiment, a peptide is from about 2 to about 100 amino acid residues in length.

A peptide can comprise any suitable L-and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a peptide can also be derivatized (e.g., alkylated) using methods known in the art.

A peptide can comprise one or more modifications (e.g., amino acid linkers, acylation, acetylation, amidation, methylation, terminal modifiers (e.g., cyclizing modifications), N-methyl-α-amino group substitution), if desired. In addition, a peptide can be an analog of a known and/or naturally-occurring peptide, for example, a peptide analog having conservative amino acid residue substitution(s).

A peptide can be linear, branched or cyclic, e.g., a peptide having a heteroatom ring structure that includes several amide bonds. Such peptides can be produced by one of skill in the art using standard techniques. For example, a peptide can be derived or removed from a native protein by enzymatic or chemical cleavage, or can be synthesized by suitable methods, for example, solid phase peptide synthesis (e.g., Merrifield-type synthesis) (see, e.g., Bodanszky et al. “Peptide Synthesis,” John Wiley & Sons, Second Edition, 1976). Peptides can also be produced, for example, using recombinant DNA methodologies or other suitable methods (see, e.g., Sambrook J. and Russell D. W., Molecular Cloning: A Laboratory Manual, 3^{rd} Edition, Cold Spring Harbor Laboratory Press, Cold Spring Harbor, New York, 2001).

As used herein, the term “antibody” is intended to encompass both whole antibodies and antibody fragments (e.g., antigen-binding fragments of antibodies, for example, Fv, Fc, Fd, Fab, Fab′, F(ab′), and dAb fragments). “Antibody” refers to both polyclonal and monoclonal antibodies and includes naturally-occurring and engineered antibodies. Thus, the term “antibody” includes, for example, human, chimeric, humanized, primatized, veneered, single chain, and domain antibodies (dAbs). (See e.g., Harlow et al., Antibodies A Laboratory Manual, Cold Spring Harbor Laboratory, 1988).

Antibodies can be produced, constructed, engineered and/or isolated by conventional methods or other suitable techniques. For example, antibodies can be raised against an appropriate immunogen, such as a recombinant mammalian (e.g., human) Gα₁₂ protein (e.g., SEQ ID NO: 1) or a portion thereof (including synthetic molecules, e.g., synthetic peptides). A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, NY); Current Protocols In Molecular Biology, Vol. 2 (Supplement 27, Summer ′94), Ausubel, F. M. et al., Eds., (John Wiley & Sons: New York, NY), Chapter 11, (1991)). Antibodies can also be raised by immunizing a suitable host (e.g., mouse) with cells that express a Gα₁₂ protein or cells engineered to express a Gα₁₂ protein (e.g., transfected cells). See e.g., Chuntharapai et al., J. Immunol., 152:1783-1789 (1994); Chuntharapai et al., U.S. Pat. No. 5,440,021. For the production of monoclonal antibodies, a hybridoma can be produced by fusing a suitable immortal cell line with antibody producing cells. The antibody producing cells can be obtained from the peripheral blood, or preferably, the spleen or lymph nodes, of humans or other suitable animals immunized with the antigen of interest. The fused cells (hybridomas) can be isolated using selective culture conditions, and cloned by limited dilution. Cells that produce antibodies with the desired specificity can be selected by a suitable assay (e.g., ELISA).

Antibody fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain. Single chain antibodies, and human, chimeric, humanized or primatized (CDR-grafted), or veneered antibodies, as well as chimeric, CDR-grafted or veneered single chain antibodies, comprising portions derived from different species, and the like are also encompassed by the present invention and the term “antibody.” The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988), regarding single chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. Cloned variable regions (e.g., dAbs) can be mutated, and sequences encoding variants with the desired specificity can be selected (e.g., from a phage library; see, e.g., Krebber et al., U.S. Pat. No. 5,514,548; Hoogenboom et al., WO 93/06213).

Other suitable methods of producing or isolating antibodies of the requisite specificity can be used, including, for example, methods which select a recombinant antibody or antibody-binding fragment (e.g., dAbs) from a library (e.g., a phage display library), or which rely upon immunization of transgenic animals (e.g., mice). Transgenic animals capable of producing a repertoire of human antibodies are well-known in the art (e.g., XENOMOUSE® (Abgenix, Fremont, CA)) and can be produced using suitable methods (see, e.g., Jakobovits et al., Proc. Natl. Acad. Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807; Lonberg et al., WO 97/13852).

As used herein, “peptidomimetic” is a molecule that is neither a peptide nor a protein, but mimics aspects of peptide or protein structure. Peptidomimetics can be prepared by conventional chemical methods (see, e.g., Damewood J. R. “Peptide Mimetic Design with the Aid of Computational Chemistry” in Reviews in Computational Biology, 2007, Vol. 9, pp. 1-80, John Wiley and Sons, Inc., New York, 1996; Kazmierski W. K., “Methods of Molecular Medicine: Peptidomimetic Protocols,” Humana Press, New Jersey, 1999). For example, a peptidomimetic can be designed by establishing the three dimensional structure of a peptide in the environment in which it is bound or will bind to a target (e.g., Gα₁₂, RhoA). A peptidomimetic comprises at least two components: a binding moiety or moieties and a backbone or supporting structure.

A binding moiety is a chemical atom or group that will react or form a complex (e.g., through hydrophobic or ionic interactions) with a target. A binding moiety in a peptidomimetic can be the same as that in a peptide or protein antagonist of the target. A binding moiety can also be an atom or chemical group that reacts with the receptor in the same or a similar manner as a binding moiety in a peptide antagonist of the target. Examples of binding moieties suitable for use in designing a peptidomimetic for a basic amino acid in a peptide include nitrogen-containing groups, such as amines, ammoniums, guanidines, amides and phosphoniums. Examples of binding moieties suitable for use in designing a peptidomimetic for an acidic amino acid include, for example, carboxyls, lower alkyl (e.g., C1-C6) carboxylic acid esters, sulfonic acids, lower alkyl sulfonic acid esters, phosphorous acids or phosphorous esters.

A supporting structure in a peptidomimetic is a chemical entity that, when bound to a binding moiety or moieties, provides the three dimensional configuration of the peptidomimetic. The supporting structure can be organic or inorganic. Examples of organic supporting structures include polysaccharides, polymers or oligomers of organic synthetic polymers (such as polyvinyl alcohol or polylactide). It is preferred that the supporting structure possess substantially the same size and dimensions as the peptide backbone or supporting structure of a peptide antagonist of a target. This can be determined by calculating or measuring the size of the atoms and bonds of a peptide and peptidomimetic. In one embodiment, a nitrogen of a peptide bond can be substituted with oxygen or sulfur, for example, forming a polyester backbone. In another embodiment, a carbonyl can be substituted with a sulfonyl group or sulfinyl group, thereby forming a polyamide (e.g., a polysulfonamide). Reverse amides of the peptide can be made (e.g., by substituting one or more —C(O)NH— groups for a —NHC(O)— group). In yet another embodiment, the peptide backbone can be substituted with a polysilane backbone.

As used herein, the term “small molecule” refers to a compound having a molecular weight of less than 1,000 daltons, for example, less than about 900 daltons, less than about 750 daltons or less than about 500 daltons. Typically, a small molecule has a molecular weight of less than about 500 daltons. Small molecules include organic compounds (e.g., steroids), organometallic compounds and inorganic compounds, and salts of organic, organometallic or inorganic compounds. Small molecules can be found in nature (e.g., identified, isolated, purified) and/or produced synthetically (e.g., by traditional organic synthesis, bio-mediated synthesis or a combination thereof). See, e.g., Ganesan, Drug Discov. Today 7(1): 47-55 (January 2002); Lou, Drug Discov. Today, 6(24): 1288-1294 (December 2001). Non-limiting examples of small molecules include rhosin, formoterol, fasudil, idelalisib and budesonide.

Also provided herein is a method of promoting relaxation of an ASMC (e.g., a HASMC), the method comprising contacting the ASMC with a Gα₁₂ inhibitor or a RhoA inhibitor (e.g., an effective amount of a Gα₁₂ inhibitor or a RhoA inhibitor). In some embodiments, the method comprises contacting the ASMC (e.g., HASMC) with a Gα₁₂ inhibitor (e.g., an effective amount of a Gα₁₂ inhibitor). In some embodiments, the method comprises contacting the ASMC (e.g., HASMC) with a RhoA inhibitor (e.g., an effective amount of a RhoA inhibitor. In some embodiments, the method comprises contacting the ASMC (e.g., HASMC) with a Gα₁₂ inhibitor (e.g., an effective amount of a Gα₁₂ inhibitor) and a RhoA inhibitor (e.g., an effective amount of a RhoA inhibitor).

“Promoting relaxation,” as used herein with respect to a cell, refers to decreasing tension of a cell, such as an ASMC (e.g., HASMC), either partially or entirely, or increasing length of a cell. A decrease in stiffness of a cell, as compared to the stiffness of the cell in its contracted state, is considered an index of single-cell smooth muscle relaxation. Stiffness can be measured by magnetic twisting cytometry, as described herein.

Also provided herein is a method of inhibiting bronchoconstriction in a subject in need thereof, comprising administering to the subject a therapeutically effective amount of a Gα₁₂ inhibitor or a RhoA inhibitor. In some embodiments, the method comprises administering to the subject a therapeutically effective amount of a Gα₁₂ inhibitor. In some embodiments, the method comprises administering to the subject a therapeutically effective amount of a RhoA inhibitor. In some embodiments, the method comprises administering to the subject a therapeutically effective amount of a Gα₁₂ inhibitor and a therapeutically effective amount of a RhoA inhibitor.

“Bronchoconstriction” refers to narrowing or tightening of the airways in the lungs. Bronchoconstriction can occur in response to an allergen or as the result of a disease, such as asthma, chronic obstructive pulmonary disease (COPD), chronic bronchitis, bronchiectasis or cystic fibrosis, and is often accompanied by coughing, wheezing and shortness of breath. In some embodiments of the methods described herein, the subject has a disease characterized by bronchoconstriction. In some embodiments of the methods described herein, the subject has airway hyperresponsiveness.

A “subject” refers to a patient who has, or is at risk for developing, bronchoconstriction or airway hyperresponsiveness or a disease characterized by bronchoconstriction. A skilled medical professional (e.g., physician) can readily determine whether a subject has, or is at risk for developing bronchoconstriction or a disease characterized by bronchoconstriction or airway hyperresponsiveness. In an embodiment, the subject is a mammal (e.g., human, non-human primate, cow, sheep, goat, horse, dog, cat, rabbit, guinea pig, rat, mouse or other bovine, ovine, equine, canine, feline or rodent organism). In a particular embodiment, the subject is a human.

Airway hyperresponsiveness is a condition characterized by a heightened sensitivity of the airways to a contractile agent, and is a feature of both asthma and chronic COPD.

Diseases characterized by bronchoconstriction include, but are not limited to, asthma, COPD, chronic bronchitis, bronchiectasis and cystic fibrosis. In some embodiments, the disease characterized by bronchoconstriction is asthma.

As used herein, a “therapeutically effective amount” is an amount that, when administered to a subject, is sufficient to achieve a desired therapeutic or prophylactic (e.g., therapeutic) effect under the conditions of administration, such as an amount sufficient to inhibit bronchoconstriction (e.g., by inhibiting Gα₁₂ or RhoaA signaling) or promote bronchodilation (e.g., by inhibiting Gα₁₂ or RhoA signaling). The effectiveness of a therapy can be determined by suitable methods known to those of skill in the art.

The amount of an inhibitor (e.g., a Gα₁₂ inhibitor, a RhoA inhibitor) to be administered (e.g., a therapeutically effective amount) can be determined by a clinician using the guidance provided herein and other methods known in the art and is dependent on several factors including, for example, the particular agent chosen, the subject's age, sensitivity, tolerance to drugs and overall well-being. Suitable dosages for antibody inhibitors can be from about 0.01 mg/kg to about 300 mg/kg body weight per treatment, from about 0.01 mg/kg to about 100 mg/kg, from about 0.01 mg/kg to about 10 mg/kg or from about 1 mg/kg to about 10 mg/kg body weight per treatment. Suitable dosages for a small molecule inhibitor can be from about 0.001 mg/kg to about 100 mg/kg, from about 0.01 mg/kg to about 100 mg/kg, from about 0.01 mg/kg to about 10 mg/kg or from about 0.01 mg/kg to about 1 mg/kg body weight per treatment. Suitable dosages for a peptide inhibitor will typically result in a plasma concentration of the peptide from about 0.1 μg/mL to about 200 μg/mL. Determining the dosage for a particular agent, patient and disease or condition is well within the abilities of one skilled in the art. Preferably, the dosage does not cause, or produces minimal, adverse side effects.

Also provided herein is a method of promoting bronchodilation in a subject in need thereof, comprising administering to the subject a therapeutically effective amount of a Gα₁₂ inhibitor or a RhoA inhibitor. In some embodiments, the method comprises administering to the subject a therapeutically effective amount of a Gα₁₂ inhibitor. In some embodiments, the method comprises administering to the subject a therapeutically effective amount of a RhoA inhibitor. In some embodiments, the method comprises administering to the subject a therapeutically effective amount of a Gα₁₂ inhibitor and a therapeutically effective amount of a RhoA inhibitor.

“Bronchodilation,” as used herein, refers to expanding (e.g., by widening or opening) the airways in the lungs. Bronchodilators, or agents that promote bronchodilation, can be useful in treating airway hyperresponsiveness or diseases associated with bronchoconstriction (e.g., asthma, COPD, chronic bronchitis, bronchiectasis, cystic fibrosis).

Also provided herein is a method of treating bronchoconstriction in a subject in need thereof, comprising administering to the subject a therapeutically effective amount of a Gα₁₂ inhibitor or a RhoA inhibitor. In some embodiments, the method comprises administering to the subject a therapeutically effective amount of a Gα₁₂ inhibitor. In some embodiments, the method comprises administering to the subject a therapeutically effective amount of a RhoA inhibitor. In some embodiments, the method comprises administering to the subject a therapeutically effective amount of a Gα₁₂ inhibitor and a therapeutically effective amount of a RhoA inhibitor.

As used herein, the terms “treat,” “treating” and “treatment” mean to counteract a medical condition (e.g., a disease characterized by bronchoconstriction or airway hyperresponsiveness, such as asthma, COPD, chronic bronchitis, bronchiectasis or cystic fibrosis) to the extent that the medical condition is improved according to a clinically-acceptable standard.

The inhibitors (e.g., Gα₁₂ inhibitors, RhoA inhibitors) described herein can be administered by a variety of routes. For example, an inhibitor can be administered by any suitable parenteral or nonparenteral route. Parenteral administration includes intraarticular, intramuscular, intravenous, intraventricular, intraarterial, intrathecal, subcutaneous and intraperitoneal administration. An inhibitor can also be administered orally, rectally, topically, by inhalation (e.g., intrabronchial, intranasal, oral inhalation or intranasal drops), nasally or ocularly. Administration can be local or systemic as appropriate, and more than one route can be used concurrently, if desired. The preferred mode of administration can vary depending upon the particular agent chosen. However, systemic intravenous or subcutaneous administration is generally preferred for antibodies. Delivery can be in vitro, in vivo, or ex vivo. In some embodiments, an inhibitor (e.g., Gα₁₂ inhibitor, RhoA inhibitor) is administered orally. In some embodiments, an inhibitor (e.g., Gα₁₂ inhibitor, RhoA inhibitor) is administered by inhalation. In some embodiments, an inhibitor (e.g., Gα₁₂ inhibitor, RhoA inhibitor) is administered nasally.

Protein inhibitors (e.g., peptides, antibodies) can be administered via in vivo expression of recombinant protein. In vivo expression can be accomplished by somatic cell expression according to suitable methods (see, e.g., U.S. Pat. No. 5,399,346). Further, a nucleic acid encoding the protein can also be incorporated into retroviral, adenoviral or other suitable vectors (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

Nucleic acid-based inhibitors (e.g., aptamers, siRNA) can be introduced into a mammalian subject of interest in a number of ways. For instance, nucleic acids may be expressed endogenously from expression vectors or PCR products in host cells or packaged into synthetic or engineered compositions (e.g., liposomes, polymers, nanoparticles) that can then be introduced directly into the bloodstream of a subject (by, e.g., injection, infusion). Anti-Gα₁₂ or RhoA nucleic acids or nucleic acid expression vectors (e.g., retroviral, adenoviral, adeno-associated and herpes simplex viral vectors, engineered vectors, non-viral-mediated vectors) can also be introduced into a subject directly using established gene therapy strategies and protocols (see, e.g., Tochilin V. P. Annu Rev Biomed Eng 8:343-375, 2006; Recombinant DNA and Gene Transfer, Office of Biotechnology Activities, National Institutes of Health Guidelines).

Typically, an inhibitor described herein (e.g., a Gα₁₂ inhibitor, a RhoA inhibitor) is administered to a subject as part of a pharmaceutical composition, for example, a pharmaceutical composition comprising the inhibitor and a pharmaceutically acceptable carrier, as described herein.

An inhibitor described herein (e.g., a Gα₁₂ inhibitor, a RhoA inhibitor) can be administered alone or as part of a combination therapy. Accordingly, in some embodiments, the methods described herein further comprise administering to the subject a therapeutically effective amount of one or more additional agents (e.g., in addition to the Gα₁₂ inhibitor or the RhoA inhibitor). In some embodiments, the additional agent is an agent for treating airway hyperresponsiveness and/or a disease characterized by bronchoconstriction. Non-limiting examples of additional agents include beta-adrenergic agonists (e.g., formoterol, salmeterol, isoproterenol), anti-inflammatory agents (e.g., budesonide, fluticasone, beclomethasone) or an agent that inhibits activation of the PI3K/ROCK axis. An additional agent can also be an agent that inhibits the M2R and/or M3R, in particular, the M3R.

Agents that inhibit activation of the PI3K/ROCK axis include, but are not limited to Gα₁₂ inhibitors, RhoA inhibitors (e.g., rhosin), agents that inhibit PI3K (e.g., idelalisib), agents that activate RhoGEF, agents that inhibit ROCK (e.g., fasudil, Y27632) and agents that inhibit MLCP. Examples of Gα₁₂ inhibitors and RhoA inhibitors include those described herein.

When an inhibitor described herein (e.g., a Gα₁₂ inhibitor, a RhoA inhibitor) is administered as part of a combination therapy, the inhibitor can be administered before, after or concurrently with the additional agent(s). In some embodiments, the Gα₁₂ inhibitor or RhoA inhibitor is administered concurrently with the additional agent(s), as either separate formulations or as a joint formulation. Alternatively, the Gα₁₂ inhibitor or RhoA inhibitor and the additional agent are administered sequentially, as separate compositions, within an appropriate time frame (e.g., a time sufficient to allow an overlap of the pharmaceutical effects of the therapies), as determined by a skilled clinician. The Gα₁₂ inhibitor or RhoA inhibitor and the additional agent(s) can be administered in a single dose or in multiple doses, in an order and on a schedule suitable to achieve a desired therapeutic effect (e.g., a reduction in bronchoconstriction). Suitable dosages and regimens of administration can be determined by a clinician and are dependent on the agent(s) chosen, pharmaceutical formulation and route of administration, various patient factors and other considerations.

### Pharmaceutical Compositions

Also provided herein is a pharmaceutical composition comprising a Gα₁₂ inhibitor (e.g., a therapeutically effective amount of a Gα₁₂ inhibitor) and a pharmaceutically acceptable carrier. In some embodiments, the therapeutically effective amount of the Gα₁₂ inhibitor is a therapeutically effective amount to treat a disease characterized by bronchoconstriction. In some embodiments, the therapeutically effective amount of the Gα₁₂ inhibitor is a therapeutically effective amount to treat airway hyperresponsiveness.

“Pharmaceutically acceptable carrier” refers to non-therapeutic components that are of sufficient purity and quality for use in the formulation of a pharmaceutical composition that, when appropriately administered to a subject (e.g., a human), do not typically produce an adverse reaction, and are used as a vehicle for a drug substance, such as a Gα₁₂ or RhoA inhibitor. “Pharmaceutically acceptable carrier” includes nontoxic, pharmaceutically acceptable carriers and/or diluents and/or adjuvants and/or excipients.

Also provided herein is a pharmaceutical composition comprising a RhoA inhibitor (e.g., a therapeutically effective amount of a RhoA inhibitor) and a pharmaceutically acceptable carrier. In some embodiments, the therapeutically effective amount of the RhoA inhibitor is a therapeutically effective amount to treat a disease characterized by bronchoconstriction. In some embodiments, the therapeutically effective amount of the RhoA inhibitor is a therapeutically effective amount to treat airway hyperresponsiveness.

In some embodiments, the pharmaceutical composition further comprises one or more additional agents (e.g., a therapeutically effective amount of one or more additional agents). In some embodiments, the additional agent(s) is an agent for treating airway hyperresponsiveness and/or a disease characterized by bronchoconstriction. In some embodiments, the pharmaceutical composition comprising an additional agent(s) comprises a therapeutically effective amount of an additional agent(s) for treating airway hyperresponsivness and/or a disease characterized by bronchoconstriction. Examples of additional agents include a beta-adrenergic agonist (e.g., formoterol, salmeterol, isoproterenol), an anti-inflammatory agent (e.g., budesonide, fluticasone, beclomethasone) or an agent that inhibits activation of the PI3K/ROCK axis (e.g., a Gα₁₂ inhibitor, such as those described herein; a RhoA inhibitor, such as those described herein; an agent that inhibits PI3K, such as idelalisib; an agent that activates RhoGEF; an agent that inhibits ROCK, such as fasudil or Y27632; an agent that inhibits MLCP). An additional agent can also be an agent that inhibits the M2R and/or M3R, in particular, the M3R. For example, a pharmaceutical composition can comprise a Gα₁₂ inhibitor and a RhoA inhibitor, such as rhosin.

The dosage form containing the pharmaceutical composition of the invention contains an amount of the active ingredient (e.g., Gα₁₂ inhibitor, RhoA inhibitor) necessary to provide a therapeutic effect. The pharmaceutical composition may contain from about 0.5 mg to about 5,000 mg (preferably, from about 0.5 mg to about 1,000 mg, more preferably, from about 0.5 mg to about 500 mg) of an inhibitor and may be constituted into any form suitable for the selected mode of administration. The composition may be administered about 1 to about 5 times per day (e.g., 1, 2, 3, 4 or 5). Daily administration or post-periodic dosing may also be employed.

The pharmaceutical compositions described herein can be formulated for administration by a variety of routes, including parenteral and nonparenteral routes. Parenteral routes includes intraarticular, intramuscular, intravenous, intraventricular, intraarterial, intrathecal, subcutaneous and intraperitoneal routes. Non-parenteral routes include oral, rectal, topical, inhalation (e.g., intrabronchial, intranasal, oral inhalation or intranasal drops), nasal and ocular routes.

In some embodiments, a pharmaceutical composition is adapted to be administered orally. Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like.

Powders can be prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules can be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, and the like. Disintegrators include, without limitation, starch, methyl cellulose, agar, bentonite, xanthan gum and the like.

Tablets can be formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acacia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit compositions for oral administration can be prolonged, delayed or sustained release formulations.

In some embodiments, a pharmaceutical composition is designed to be administered by inhalation. Dosage forms for inhaled administration may conveniently be formulated as aerosols or dry powders, which may be generated by means of various types of metered, dose pressurized aerosols, nebulizers or insufflators.

Aerosol formulations, e.g., for inhaled administration, can comprise a solution or fine suspension of an inhibitor in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively, the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the inhibitor. This may require the incorporation of additional excipients, e.g., co-solvents and/or surfactants, to improve the dispersion characteristics and homogeneity of suspension formulations. Solution formulations may also require the addition of co-solvents such as ethanol.

Dry powders adapted for administration by inhalation can comprise a powder base, such as lactose, glucose, trehalose, mannitol or starch, an inhibitor and optionally a performance modifier, such as L-leucine or another amino acid, and/or metal salts of stearic acid, such as magnesium or calcium stearate.

In some embodiments, a pharmaceutical composition is designed to be administered nasally. Dosage forms for nasal administration may conveniently be formulated as aerosols, solutions, drops, gels or dry powders. For pharmaceutical compositions suitable and/or adapted for intranasal administration, an inhibitor can be formulated as a fluid formulation for delivery from a fluid dispenser. Such fluid dispensers may have, for example, a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity.

### Screening Methods

Also provided herein is a method of identifying an agent that inhibits contraction and/or promotes relaxation of an ASMC (e.g., HASMC). The method comprises contacting an ASMC with a contractile agent and a candidate agent that inhibits contraction or promotes relaxation of an ASMC and measuring activation of the PI3K/ROCK axis in the ASMC. A reduction in activation of the PI3K/ROCK axis in an ASMC that has been contacted with the candidate agent compared to a control indicates that the candidate agent inhibits contraction or promotes relaxation of an ASMC. In some embodiments, the method is a method of identifying an agent that inhibits contraction of an ASMC (e.g., HASMC), comprising contacting an ASMC with a contractile agent and a candidate agent that inhibits contraction of an ASMC, and measuring activation of the PI3K/ROCK axis in the ASMC, wherein a reduction in activation of the PI3K/ROCK axis in an ASMC that has been contacted with the candidate agent compared to a control indicates that the candidate agent inhibits contraction of an ASMC. In some embodiments, the method is a method of identifying an agent that promotes relaxation of an ASMC (e.g., HASMC), comprising contacting an ASMC with a contractile agent and a candidate agent that promotes relaxation of an ASMC, and measuring activation of the PI3K/ROCK axis in the ASMC, wherein a reduction in activation of the PI3K/ROCK axis in an ASMC that has been contacted with the candidate agent compared to a control indicates that the candidate agent promotes relaxation of an ASMC.

A “contractile agent” is an agent that induces or promotes contraction of a cell, particularly an ASMC (e.g., HASMC). Contractile agents include, but are not limited to, carbachol, histamine, thrombin, methacholine, acetylcholine and lysophosphatidic acid (LPA). Other contractile agents will be known to one of skill in the art.

In some embodiments, the screening methods described herein are conveniently conducted in multi-well plate format using, for example, cultured ASMCs (e.g., HASMCs) or lung tissue (e.g., hPCLS).

Candidate agents that inhibit contraction or promote relaxation of an ASMC include, for example, nucleic acids (e.g., siRNA), peptides (e.g., a polypeptide comprising a regulator of G-protein signaling (RGS) domain), antibodies, peptidomimetics and small molecules (e.g., rhosin).

In some embodiments, measuring activation of the PI3K/ROCK axis comprises measuring phosphorylation of AKT, myosin phosphatase targeting subunit-1 (MYPT1) and/or myosin light chain-20 (MLC). Representative methods of measuring phosphorylation of AKT, MYPT1 and MLC are described in the Exemplification herein.

In some embodiments, measuring activation of the PI3K/ROCK axis comprises measuring reporter expression, such as luciferase expression, for example, of a serum response element (SRE)-luciferase reporter construct that induces luciferase expression upon Gα₁₂ activation. Representative methods of measuring reporter expression are described in the Exemplification herein.

### EXEMPLIFICATION

### Methods

Materials. CHRM2 (L-005463-01-0005), CHRM3 (L-005464-00-0005), NT siRNA (D-001810-10-05), GNA12 (L-008435-00-0005), GNA13 (L-009948-00-0005), RhoA (L-003860-00-0005), and Rac1 (L-003560-00-0005) siRNAs were obtained from Dharmacon (Lafayette, CO, USA). Carbachol (carbamoyl choline chloride), formoterol (formoterol fumarate dihydrate), isoprenaline (ISO—isoproterenol hydrochloride), bradykinin (bradykinin acetate salt), pertussis toxin and perchloric acid were purchased from Sigma Aldrich (St. Louis, MO, USA). Rhosin (555460) was purchased from EMD Millipore (Darmstadt, Germany). Antibodies for detection of pMYPT1-Thr696 (5163S), pAkt (4060S), pMLC (3674S) and GAPDH (2118S), total AKT (4691S) were purchased from Cell Signaling Technologies (Danvers, MA, USA). Antibodies for immunoprecipitation and detection of the M3 receptor (SC-9108) and Gα₁₂ (SC-409) were obtained from Santa Cruz Biotechnology (Dallas, TX, USA). Total MLC antibody (MABT180) was obtained from EMD Millipore (Darmstadt, Germany). Total MYPT1 antibody (612165) was obtained from BD Biosciences (San Jose, CA, USA).

Isolation and culture of HASMC. Human lungs were received from the National Disease Research Interchange (Philadelphia, PA, USA) and from the International Institute for the Advancement of Medicine (Edison, NJ, USA) and HASM cells were derived from the tracheas. All cell lines and tissue are obtained from de-identified donors and their use does not constitute human subject research as described by the Rutgers Institutional Review Board. Culture of HASM cells was conducted as described previously (Panettieri et al., 1989a). Briefly, cells were cultured in Ham's F-12 medium supplemented with 100 U mL⁻¹ penicillin, 0.1 mg mL⁻¹, streptomycin, 2.5 mg mL⁻¹ amphotericin B and 10% FBS. Medium was replaced every 72 hours. HASM cells were only used during subculture passages 1-4 due to the strong expression of native contractile proteins (Panettieri et al., 1989b). In pertussis toxin studies, cells were treated with 1 μg/ml of pertussis toxin for 18 hours. All pharmacologic inhibitors were used with DMSO as the vehicle at a final concentration of 0.1% and were used to treat HASMC 30 minutes prior to agonist stimulation.

Retroviral Infection. Stable expression of GFP and p115rhogefRGS-GFP was achieved by retroviral infection as described previously (Kong et al., 2008; Deshpande et al., 2014). Briefly, retrovirus for the expression of each construct was produced by cotransfecting GP2-293 cells with pVSV-G vector (encoding the pantropic (VSV-G) envelope protein) and pLPCX-GFP or pLPCX-p115rhogefRGS-GFP. Forty-eight hours after transfection, supernatants were harvested and used to infect human telomerase reverse transcriptase (hTERT) immortalized airway smooth muscle cultures, with effective virus concentrations established by immunoblot analysis. Cultures were selected to homogeneity with 1 μg mL⁻¹ puromycin, as described previously (Kong et al., 2008; Deshpande et al., 2014).

Generation of hPCLS and airway dilation assays. hPCLS were prepared as previously described (Cooper et al., 2009). Briefly, human lungs were dissected and filled with 2% (w v⁻¹) low melting point agarose. After the agarose solidified, the lobe was sectioned and 8 mm diameter cores were generated. Cores containing small airways were sliced at a thickness of 350 μM using Precisionary Instruments VF300 Vibratome. They were then collected in supplemented Ham's F-12 medium. Generated slices came from all areas of the lung and not just one specific area. Airways from each core were randomized to the different treatment groups prior to the start of the experiment. Airways were constricted to a dose response of carbachol (10⁻⁸-10⁻⁵ M), then dilated to one of the following (10⁻¹¹-10⁻⁴ M): diluent (DMSO), formoterol, or rhosin. DMSO alone did not induce airway dilation at the concentrations tested (data not shown).

To assess luminal area, lung slices were placed in a 12-well plate in media and held in place using a platinum weight with nylon attachments. The airway was located using a microscope (Nikon Eclipse; model no. TE2000-U; magnification, ×40) connected to a live video feed (Evolution QEi; model no. 32-0074A-130 video recorder). Airway luminal area was measured using Image-Pro Plus software (version 6.0; Media Cybernetics) and represented in mm² (Cooper et al., 2009). After functional studies, the area of each airway at baseline and at the end of dose the response was calculated using Image-Pro Plus software. Maximal effect of drug (Eₘₐₓ), log of the concentration to induce 50% of maximal drug effect (log EC₅₀) and the area under the curve (AUC), were calculated from the dose-response curves. Airway dilation was calculated as percent (%) reversal of maximal bronchoconstriction and expressed as % forskolin response after normalizing to forskolin stimulation (10 μM).

siRNA transfection. Ham's F-12 media, DharmaFECT 1 reagent, and siRNA were combined in a microcentrifuge tube according to manufacturer's protocol and incubated for 20 minutes. HASMCs were trypsinized and trypsin was inactivated with 5% FBS. Cells were centrifuged and resuspended in Ham's F-12 media. Cell suspension was added to siRNA mixture and incubated for 15 minutes. Cell suspension and siRNA mixture was then seeded into cell culture plates according to experimental design and incubated for 6 hours. After 6 hours, complete cell culture media (described above) was added to the cell culture plate wells in a 1:1 ratio and was incubated for 18 hours. After 18 hours, media was changed to complete media. Cells were serum-deprived for 24 hours before collection. Cells were collected 72 hours post-transfection.

cAMP Assay. HASMCs were seeded in a 24-well plate until about 80% confluent and serum-deprived overnight. Cells were stimulated and lysed using cAMP-Screen System ELISA from Applied Biosystems (Bedford, MA, USA). Experiment was conducted according to manufacturer's protocol.

SRE-Luciferase Assay. HASMC were seeded and grown to 75% confluence. Complete medium was removed and Cignal Lenti SRE Reporter (CLS-010L-1) was added to cells with SureENTRY Transduction reagent (336921) according to manufacturer's protocol. After 24 hours, media was changed to complete medium. After 24 hours, media was changed to serum-free media. Following 48 hours incubation in serum-free media, cells were stimulated with carbachol for 6 hours and collected with luciferase lysis buffer (E1483) from Promega (Madison, WI, USA).

Immunoblot analysis. After transfection with siRNA or incubation with pharmacologic inhibitors, cells were stimulated with carbachol (10 μM-10 minutes). Perchloric acid was added to cell media to attain a final concentration of 0.1%. Cells were scraped, collected, and pelleted. Pellets were washed once with ice-cold PBS. PBS was aspirated and pellets were solubilized in RIPA. Sample buffer was added and samples were subjected to SDS-PAGE and transferred to nitrocellulose membranes, as previously described (Balenga et al., 2015; Koziol-White et al., 2016). Phosphorylation of MYPT1, MLC and AKT were assessed, and band densities were normalized to GAPDH, total MYPT1, total MLC, or total AKT band density.

Co-immunoprecipitation. After stimulation, HASMCs grown on 10 cm plates were lysed using ice-cold cell lysis buffer from Cell Signaling Technology (Danvers, MA, USA) containing 1% Triton X-100 with Protease and Phosphatase Inhibitors from Thermo Fisher Scientific (Waltham, MA, USA). Lysate was incubated with primary antibody and incubated overnight with gentle rocking at 4° C. Protein A was incubated with lysates with gentle rocking for 3 hours at 4° C. Samples were microcentrifuged for 30 seconds at 4° C. Pellet was washed five times with cell lysis buffer. Pellet was resuspended with SDS sample buffer and heated for 10 minutes at 70° C. Sample was then loaded onto SDS-PAGE gel and analyzed by immunoblot.

Magnetic Twisting Cytometry. Dynamic changes in cell stiffness were measured in isolated human ASM using forced motions of functionalized beads anchored to the cytoskeleton through cell surface integrin receptors, as previously described in detail (Fabry et al., 2001; An et al., 2006; Deshpande et al., 2010). The increase or decrease in stiffness is considered an index of single-cell smooth muscle contraction and relaxation, respectively. For these studies, serum-deprived, postconfluent cultured ASM cells were plated at 30,000 cells/cm² on plastic wells (96-well Removawell, Immulon II; Dynatec Labs, El Paso, TX) previously coated with type I collagen (VitroCol; Advanced BioMatrix, Inc., San Diego, CA) at 500 ng/cm², and maintained in serum-free media for 24 hours at 37° C. in humidified air containing 5% CO₂. These conditions have been optimized for seeding cultured cells on collagen matrix and for assessing their mechanical properties. For each individual cell, the baseline stiffness was measured for the first 60 seconds, and after drug addition, the stiffness was measured continuously for the next 15 minutes. Drug-induced changes in cell stiffness approached a steady-state level by 15 minutes. Agonist-induced contraction was normalized to baseline contraction and expressed as % over basal.

Micro-pattern Deformation. Soft silicone elastomer films were micro-patterned with fibronectin and fluorescent fibrinogen in uniform ‘X’ shapes (70 μm diagonal by 10 μm thick) as previously described (Tseng et al., 2014; Koziol-White et al., 2016). The non-patterned regions were blocked using 0.5% Pluronic F-127 inhibiting cellular adhesion away from the fibronectin patterns. Isolated cells adhering to these ‘X’-shaped micro-patterns exerted traction forces causing deformations of the micro-patterns. Dimensions of contracted micro-patterns, which correspond directly to the force applied on them by adhered cells, relative to the original unperturbed dimensions were used to assess cellular contractile responses to carbachol. Prior to stimulation, cells were seeded into a 96-well plate functionalized with the described micropatterned elastomeric film (into 36 wells each), allowed to adhere and serum-starved for 24 hours. At the time of the experiment, cells were imaged at baseline, treated with carbachol (30 μM) and imaged at five 6-minute intervals, then treated with bradykinin (10⁻⁵ M) and imaged for an addition four 6-minute intervals. Cell nuclei were stained with Hoechst 33342 prior to imaging and only the patterns co-localized with exactly one stained nucleus were used in the analysis. Following these studies, MATLAB was used to measure each individual pattern occupied by a single cell at each interval. Using an additional automated script, each population was mined for ‘responder’ cells, defined as the individual cells that exhibited at least a 25% percent contractile increase over baseline at their peak response to bradykinin (which acts via an orthogonal pathway to carbachol). The contractile activity to carbachol was compared among such responders from each group.

Statistical analysis. The data and statistical analysis comply with the recommendations on experimental design and analysis in pharmacology (Curtis et al., 2015). Each experimental condition was normalized to the basal condition (fold/basal) for each statistical replicate before taking averages. For experiments comparing agonist responses in p115RhoGEF-RGS-expressing cell lines to control cell lines, agonist response was normalized to basal response in each respective cell line (fold/basal) and subsequently analyzed statistically. For experiments comparing agonist responses in primary cells, conditions were all normalized to the basal condition and subsequently analyzed statistically. GraphPad Prism software (La Jolla, CA, USA) was used to determine statistical significance evaluated by Student's unpaired t-test for two groups or ANOVA with Bonferroni post-test for 3 or more groups. P values of <0.05 were considered significant. For single cell shortening data, cells were not compared with themselves for each treatment group, so repeated measures analysis was not used. Data were normally distributed, and ANOVAs were used for data analysis, with Bonferroni's post-test. Differences were isolated using the Bonferroni post-test for all pairwise comparisons. Magnetic twisting cytometry data were analyzed by Student's two-tailed t-tests. SigmaStat (Systat, San Jose, CA, USA) and GraphPad Prism software were used in statistical analyses.

### Results

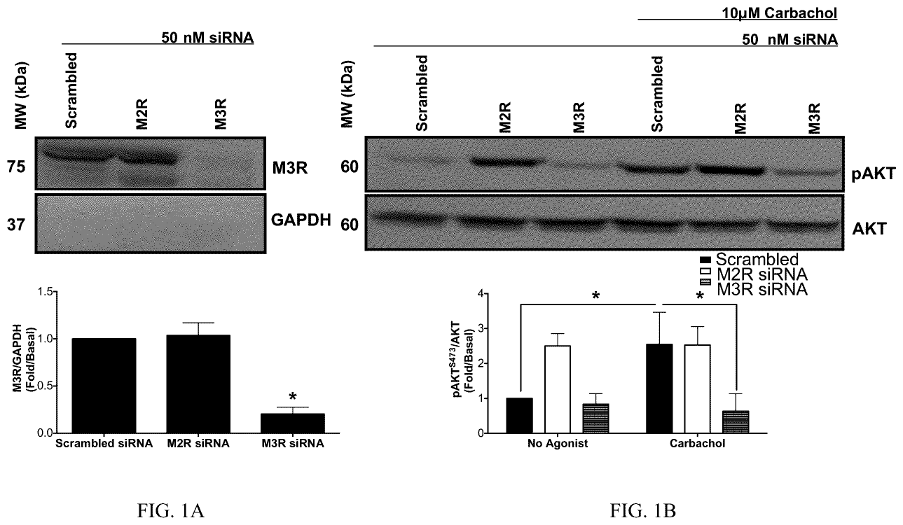

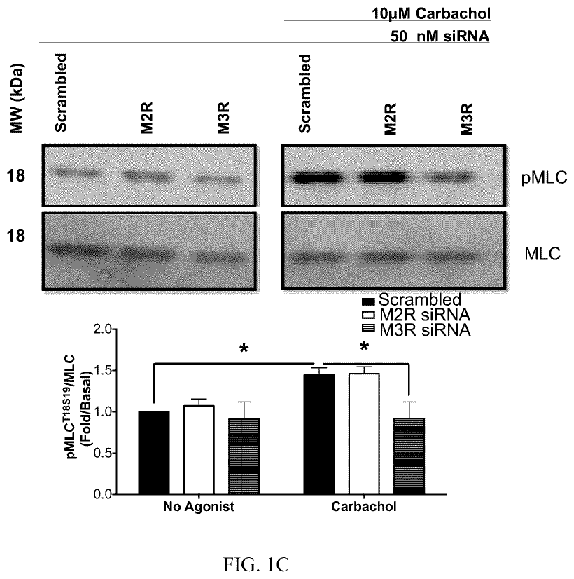

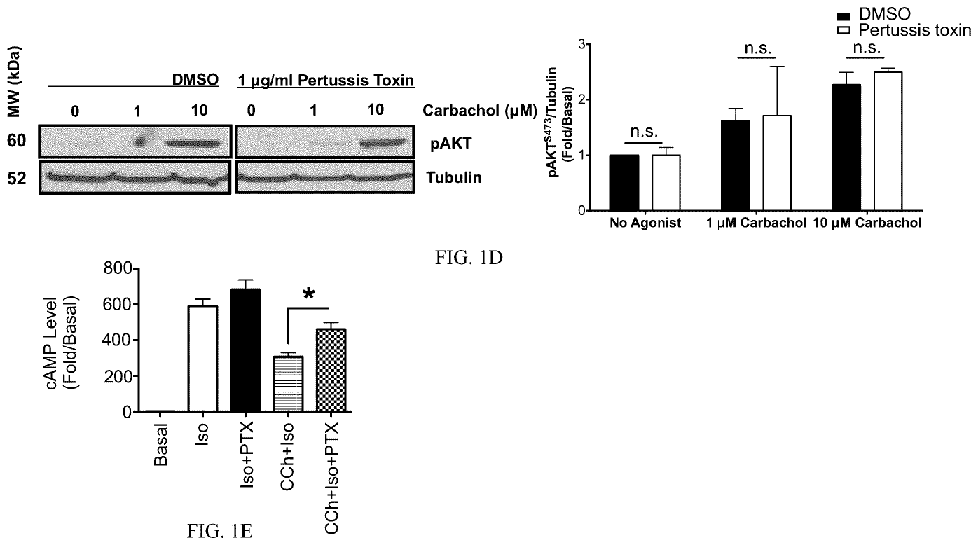

The M3 muscarinic receptor, not the M2 muscarinic receptor, mediates carbachol-induced AKT and MLC phosphorylation. The M2 and M3 muscarinic receptor subtypes are expressed in HASMCs (Billington and Penn, 2002). In order to determine the receptor(s) contributing to carbachol-mediated activation of PI3K/ROCK axis, the effects of carbachol stimulation (10 μM, 10 min) on AKT (S473) and MLC (S19) phosphorylation in primary HASMCs 72 hours after transfection were studied with M2R, M3R, or scrambled siRNA. Immunoblot analysis confirmed M3R siRNA reduced M3R protein expression (80±7%) (FIG. 1A), whereas M2R knockdown was confirmed by quantitative PCR. M3R siRNA attenuated carbachol-induced phosphorylation of AKT (2.5±0.9 fold vs. 0.6±0.4 fold) and phosphorylation of MLC (1.4±0.1 fold vs. 0.3±0.3 fold) compared to scrambled siRNA (FIGS. 1B and 1C). M2R siRNA had little effect on carbachol-induced MLC phosphorylation when compared to scrambled siRNA (1.4±0.1 fold vs 1.4±0.1 fold). Surprisingly, M2R siRNA induced AKT phosphorylation (2.5±0.4 fold) in the absence of agonist. Since the M2R couples predominantly to the G protein Gai, pertussis toxin (18 h, 1 μg m⁻¹) was used to ADP-ribosylate Gai, rendering it inactive, and AKT phosphorylation was measured in response to carbachol stimulation (10 μM, 10 minutes). Incubation with pertussis toxin before carbachol stimulation had little effect on AKT phosphorylation when compared to vehicle (0.01% DMSO) (FIG. 1D), yet rescued carbachol-induced attenuation of isoproterenol-mediated cAMP elevation (306±23 fold without PTX vs. 461±37 fold with PTX) (FIG. 1E), suggesting the effect of M2R knockdown was independent of any reduction in M2R activation of Gαᵢ.

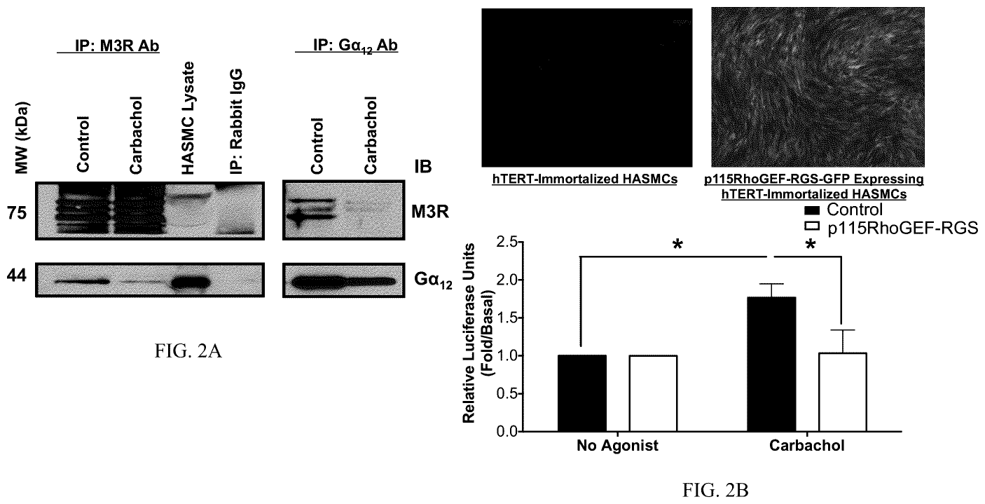

Gα₁₂ couples to the M3R in HASMCs. Previous reports in HEK293 cells using GTP photolabelling and Gα₁₂-specific RGS overexpression demonstrate that Gα₁₂ is coupled to the M3R (Rümenapp et al., 2001; Riobo and Manning, 2005). To determine whether coupling occurs in HASMCs co-immunoprecipitation techniques were used to pull down the M3R and Gα₁₂ proteins. These samples were subsequently immunoblotted for the indicated proteins (FIG. 2A). When the M3R was immunoprecipitated and subsequently probed with Gα₁₂ antibody, a strong band was present for Gα₁₂. In HASMCs subject to carbachol stimulation (10 μM, 1 minute), the band density diminished. Interestingly, when Gα₁₂ was immunoprecipitated and subsequently immunoblotted using the M3R antibody, a strong band was also present. Again, band density diminished under conditions of carbachol stimulation. To further evaluate M3R-Gα₁₂ coupling, hTERT-immortalized HASMCs overexpressing a GFP-tagged RGS domain of the p115RhoGEF enzyme (p115RhoGEF-RGS-GFP) were infected with an SRE-luciferase reporter construct that induces luciferase expression upon Gα₁₂ activation (FIG. 2B). These cells were stimulated with carbachol (10 μM, 6 hours), lysed, and assayed for luciferase induction using luminescence. Carbachol stimulation elevated luciferase expression (76±18% fold). Carbachol-induced luciferase induction was reduced to basal levels in HASMCs expressing p115RhoGEF-RGS (76±18 fold vs. 1.0±0.3 fold with PTX), suggesting the effective inhibition of Gα₁₂ signaling by p115RhoGEF-RGS-GFP.

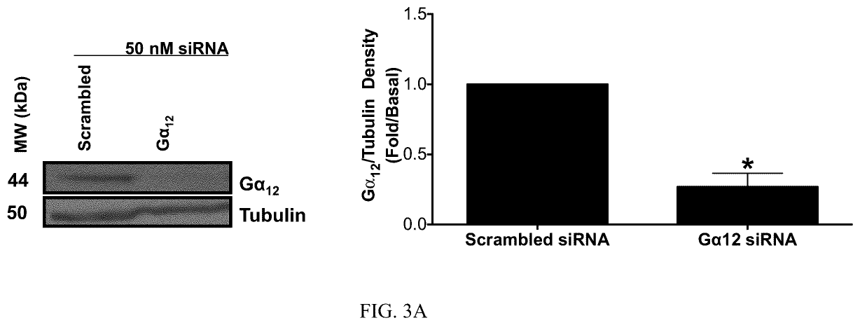

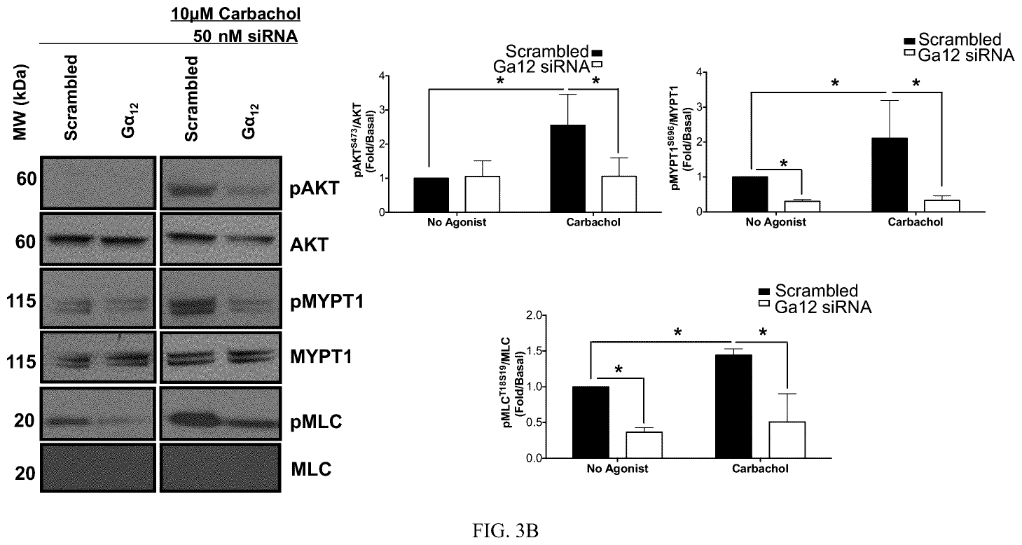

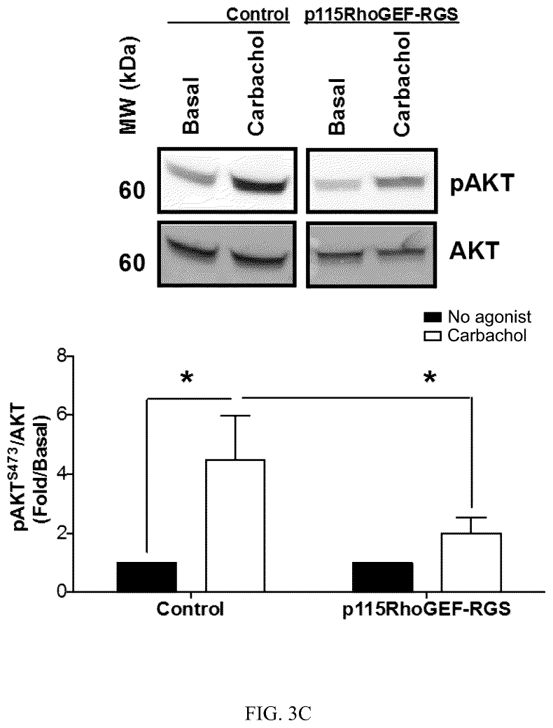

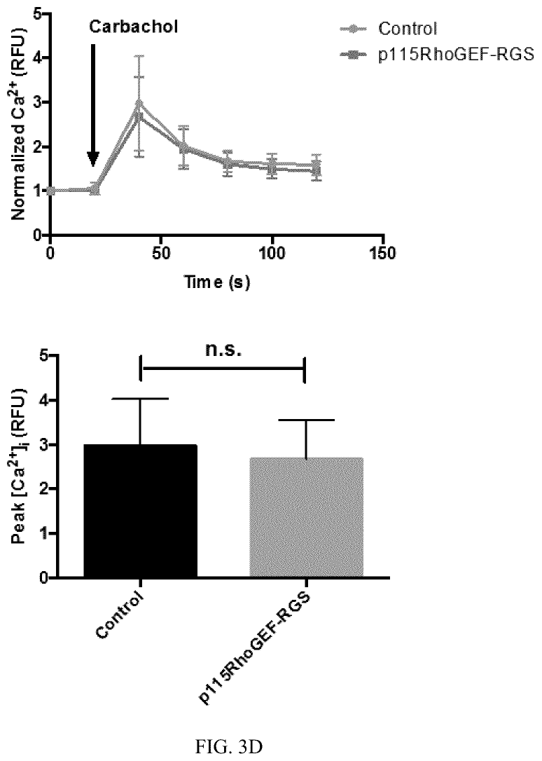

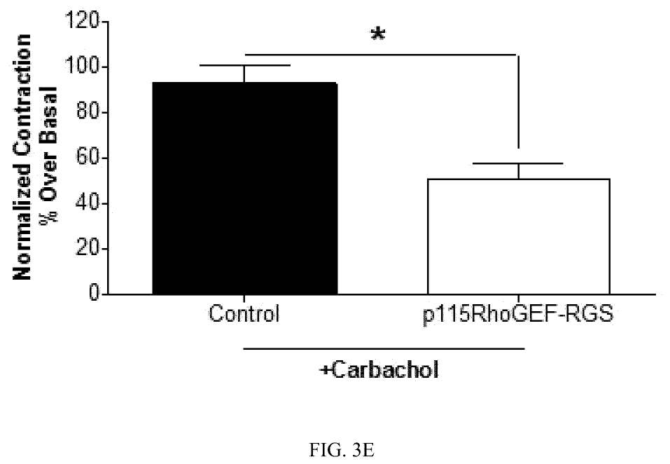

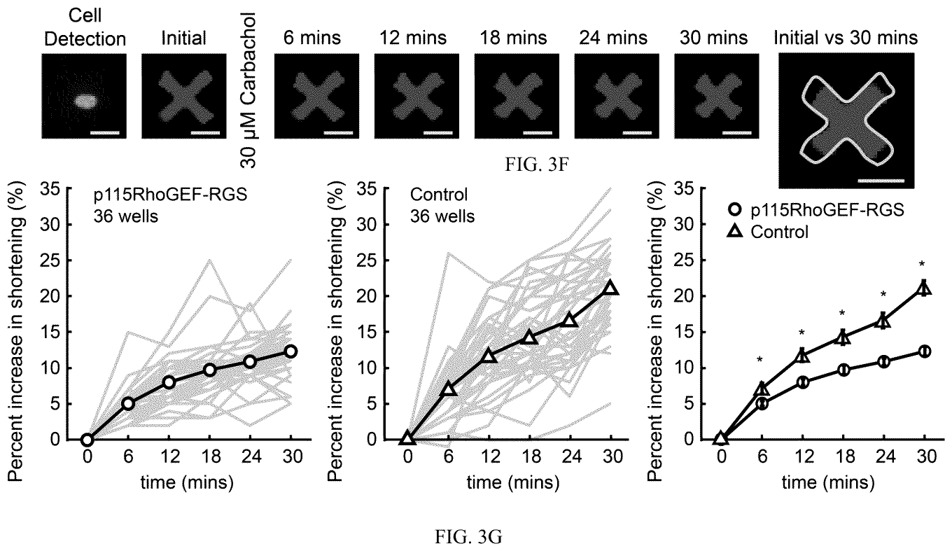

Gα₁₂ mediates M3R-induced activation of PI3K/ROCK axis activation. To determine the contribution of Gα₁₂ proteins to carbachol-induced PI3K/ROCK axis activation, siRNA was used to knockdown Gα₁₂ proteins and the effects on carbachol-induced (10 μM, 10 minutes) phosphorylation of AKT, MYPT1, and MLC in primary HASMCs were measured 72 hours after transfection with Gα₁₂ proteins or scrambled siRNA. Gα₁₂ siRNA knockdown reduced Gα₁₂ protein expression (73±9%) (FIG. 3A) and scrambled siRNA had little effect on any of the proteins examined. Gα₁₂ siRNA markedly attenuated carbachol-induced phosphorylation of AKT (2.5±0.9 fold vs 1.0±0.5 fold), phosphorylation of MYPT1 (2.1±1.0 fold vs 0.3±0.1 fold), and phosphorylation of MLC (1.4±0.1 fold vs 0.5±0.4 fold) compared to scrambled siRNA (FIG. 3B). To complement the Gα₁₂ siRNA studies, carbachol-induced AKT phosphorylation and contraction in hTERT-immortalized HASMC that do/do not express p115RhoGEF-RGS was compared. In p115RhoGEF-RGS-expressing HASMCs, carbachol-induced AKT phosphorylation was attenuated compared to control cell lines (2±0.5 fold vs. 4.5±1.5 fold) (FIG. 3C). p115RhoGEF-RGS expression had little effect on Gα_{q} activation, as measured by intracellular calcium mobilization (FIG. 3D). Carbachol-induced contraction and shortening were also attenuated compared to control cell lines (92.8±7.9% vs. 50.4±7.1%) (FIGS. 3E and 3G).

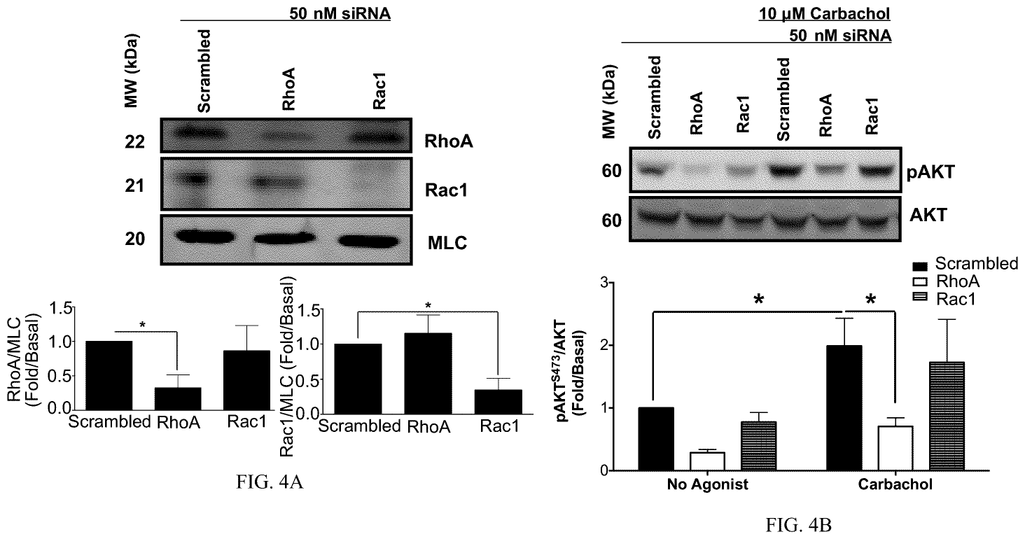

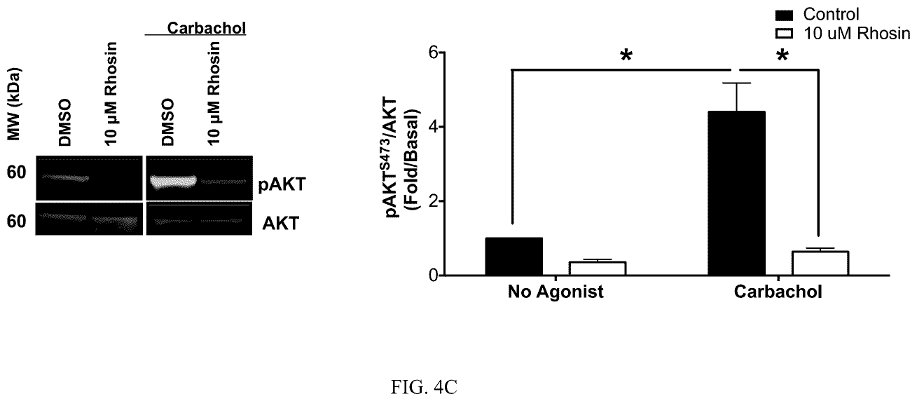

Gα₁₂-mediated activation of PI3K is RhoA-dependent. Whereas previous studies have implicated PI3K in the activation of Rho kinase by carbachol, the potential for Rho family GTPases to regulate PI3K isoforms has been previously suggested (Yang et al., 2012). In order to determine whether Gα₁₂-mediated activation of PI3K involved Rho and Rac small GTPases as signaling intermediates, the effects of carbachol stimulation (10 μM, 10 minutes) on AKT (S473) phosphorylation in primary HASMCs were examined 72 hours after transfection with RhoA, Rac1 or scrambled siRNA. RhoA and Rac1 siRNA knockdown reduced protein expression (68±19% and 66±16% respectively) (FIG. 4A) and scrambled siRNA had little effect on any of the proteins examined. RhoA siRNA attenuated carbachol-induced phosphorylation of AKT (2±0.4 fold vs. 0.7±0.1 fold) compared to scrambled siRNA (FIG. 4B). To complement siRNA studies, rhosin was used to inhibit RhoGEFs that activate RhoA and AKT phosphorylation in response to carbachol stimulation was measured. Incubation with rhosin attenuated carbachol-induced phosphorylation of AKT (4.4±0.8 fold vs. 0.6±0.1 fold) compared to vehicle (FIG. 4C). These results suggest that RhoA either functions upstream of PI3K, or modulates activation of PI3K through cooperativity

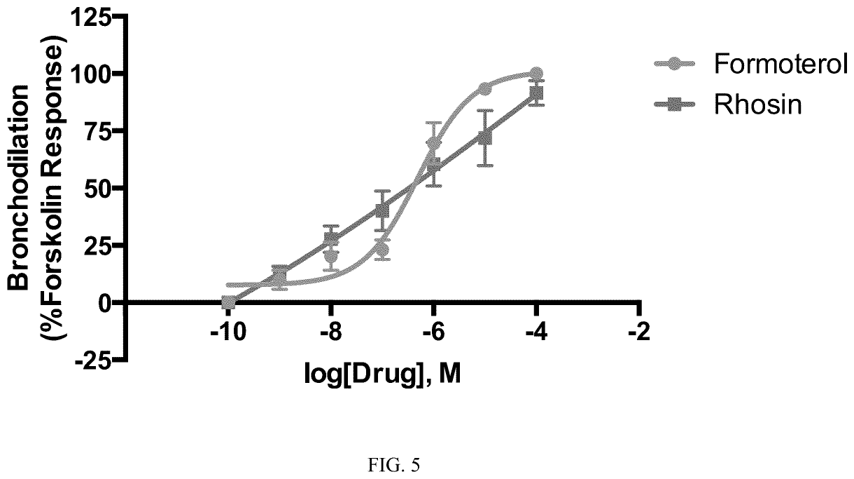

RhoA inhibition promotes bronchodilation of hPCLS. To determine if inhibition of RhoA reverses agonist-induced bronchoconstriction in hPCLS, hPCLS were stimulated with carbachol to induce luminal narrowing and subsequently treated with increasing doses of rhosin or formoterol to evaluate airway dilation (FIG. 5). Formoterol reversed carbachol-induced bronchoconstriction with an Emax of 100±3% and log EC₅₀ of −6.3.

### DISCUSSION

This study demonstrates a previously unidentified role for Gα₁₂ in modulating M3R-mediated activation of the PI3K/ROCK axis in HASMCs. The study also demonstrates that Gα₁₂-mediated activation of PI3K/ROCK axis is RhoA-dependent. Furthermore, the study shows that inhibition of RhoA blunts carbachol-induced PI3K activation and promotes bronchodilation of human small airways, implicating RhoA as a pivotal mediator of airway tone.

siRNA and pharmacological tools, as well as HASMCs overexpressing p115RhoGEF-RGS proteins that inhibit M3R-mediated activation of Gα₁₂ were used to determine the role of Gα₁₂ in modulating PI3K/ROCK axis activation and HASMC contraction. The data show that knockdown of the M3R attenuated carbachol-induced activation of AKT, MYPT1, and MLC phosphorylation. The data also show that Gα₁₂ coimmunoprecipitated with the M3R, and that p115RhoGEF-RGS expression inhibits carbachol-mediated induction of SRE-luciferase reporter. Gα₁₂ siRNA attenuated carbachol-induced activation of AKT, MYPT1, and MLC phosphorylation, and p115RhoGEF-RGS overexpression similarly reduced carbachol-induced activation of AKT and HASM contraction. Furthermore, it was demonstrated that siRNA and pharmacological inhibition of RhoA blunted carbachol-mediated activation of PI3K, and RhoA inhibitors induced dilation of hPCLS, implicating RhoA as a pivotal mediator of airway tone.

Despite its lower expression levels, investigators suggest that the Gα_{q}-coupled M3 muscarinic receptor, and not the Gai-coupled M2 muscarinic receptor, is the primary subtype responsible for bronchial and tracheal smooth muscle contraction (Roffel et al., 1988, 1990; van Nieuwstadt et al., 1997; Murthy et al., 2003; Fisher et al., 2004). Nonetheless, some studies suggest a role for the M2R in mediating airway smooth muscle contraction in the peripheral airways (Roffel et al., 1993; Struckmann et al., 2003). These findings using siRNA against the M2R and M3R siRNA, as well as pertussis toxin to inactivate M2R-coupled Gai demonstrate that the M3R is the dominant receptor mediating the activation of the PI3K/ROCK axis (FIG. 1). Incubation with M2R siRNA surprisingly resulted in a robust activation of PI3K that possibly could be related to compensatory expression of proteins that activate PI3K (Murthy et al., 2003). These data stand in contrast with studies conducted in rabbit intestinal smooth muscle, where the M2R through Gβγ-dependent signaling, activates PI3K (Murthy et al., 2003). Interestingly, the rabbit intestinal smooth muscle cells expressed p110γ isoform of PI3K that is not expressed in the HASMCs used in these studies (Goncharova et al., 2002; Jude et al., 2012; Himes et al., 2015; Koziol-White et al., 2016). Gβγ proteins are typically thought to signal to the p110γ isoforms of PI3K, not the p110α, p110β, or p110δ isoforms expressed in the HASMCs used herein (Leopoldt et al., 1998). This illustrates an important concept that the identical receptors mediate signaling that is tissue and species specific.

GPCR-mediated activation of PI3K can occur through epidermal growth factor receptor (EGFR) transactivation (Wang, 2016). Previous studies, however, have demonstrated a lack of EGFR phosphorylation induced by carbachol in HASMCs (Krymskaya et al., 2000).

Since Gα_{12/13} family proteins have been shown to modulate RhoA/ROCK pathways in other cell types, co-immunoprecipitation and SRE-luciferase reporter expressing HASMCs were used to demonstrate whether the M3R coupled to Gα₁₂ in HASMCs (FIG. 2). The results disclosed herein suggest that the M3R indeed is coupled to Gα₁₂ in HASMCs and that M3R-induced activation of Gα₁₂ is attenuated by overexpression of the p115RhoGEF-RGS domain. Furthermore, Gα₁₂ siRNA attenuated carbachol-induced AKT, MYPT1, MLC phosphorylation, suggesting that Gα₁₂ regulates PI3K/ROCK axis activation and MLC phosphorylation in HASMCs. These data agree with previous studies demonstrating M3R-Gα₁₂ coupling in HEK293 cells (Rümenapp et al., 2001). Other studies have suggested a lack of M3R-Gα₁₂ coupling in murine airway smooth muscle; however, these findings may be a result of species differences between mice and humans. The data disclosed herein highlight the importance and necessity of Gα₁₂ proteins in maintaining HASMC tone through pathways involving the PI3K-ROCK axis.

In order to determine whether Gα₁₂-mediated activation of the PI3K-ROCK axis involved RhoA, RhoA siRNA and rhosin, a rationally design inhibitor of RhoA, were used to test whether limiting RhoA signaling would attenuate PI3K activation. The data disclosed herein show that RhoA siRNA and inhibitors attenuated carbachol-induced AKT phosphorylation, suggesting PI3K as an intermediate in Gα₁₂ signaling. The RhoA siRNA data disclosed herein, in particular, support the idea that Gα₁₂-mediated activation of PI3K is RhoA-dependent.

Using hPCLS, it was demonstrated that RhoA inhibition by rhosin induced bronchodilation was comparable to formoterol, an industry standard bronchodilator, suggesting that inhibition of Gα₁₂-mediated signaling pathway provides an alternative therapeutic strategy for bronchodilation in asthma.

The data described herein demonstrate coupling of the M3R to Gα₁₂ in HASMCs and that Gα₁₂ plays a role in contraction through RhoA-dependent activation of the PI3K/ROCK axis. Inhibition of RhoA induces bronchodilation in hPCLS.

### REFERENCES

Amrani, Y., Tliba, O., Deshpande, D. A., Walseth, T. F., Kannan, M. S., and Panettieri, R. A. (2004). Bronchial hyperresponsiveness: Insights into new signaling molecules. Curr. Opin. Pharmacol. 4: 230-234.
An, S. S., Fabry, B., Trepat, X., Wang, N., and Fredberg, J. J. (2006). Do Biophysical Properties of the Airway Smooth Muscle in Culture Predict Airway Hyperresponsiveness?Am. J. Respir. Cell Mol. Biol. 35: 55-64.
Balenga, N. a., Klichinsky, M., Xie, Z., Chan, E. C., Zhao, M., Jude, J., et al. (2015). A fungal protease allergen provokes airway hyper-responsiveness in asthma. Nat. Commun. 6: 6763.
Billington, C. K., and Penn, R. B. (2002). M3 muscarinic acetylcholine receptor regulation in the airway. Am J Respir Cell Mol. Biol 26: 269-272.
Billington, C. K., and Penn, R. B. (2003). Signaling and regulation of G protein-coupled receptors in airway smooth muscle. Respir. Res. 4: 2.
Black, J. L., Panettieri, R. A., Banerjee, A., and Berger, P. (2012). Airway Smooth Muscle in Asthma. Just a Target for Bronchodilation?Clin. Chest Med. 33: 543-558.
Bradley, S. J., Wiegman, C. H., Iglesias, M. M., Kong, K. C., Butcher, A. J., Plouffe, B., et al. (2016). Mapping physiological G protein-coupled receptor signaling pathways reveals a role for receptor phosphorylation in airway contraction. Proc. Natl. Acad. Sci. U.S.A 113: 4524-4529.
Chiba, Y., Matsusue, K., and Misawa, M. (2010). RhoA, a possible target for treatment of airway hyperresponsiveness in bronchial asthma. J. Pharmacol. Sci. 114: 239-247.
Chiba, Y., and Misawa, M. (2001). Increased expression of G12 and G13 proteins in bronchial smooth muscle of airway hyperresponsive rats. Inflamm. Res. 50: 333-6.
Chiba, Y., and Misawa, M. (2004). The role of RhoA-mediated Ca2+ sensitization of bronchial smooth muscle contraction in airway hyperresponsiveness. J. Smooth Muscle Res. 40: 155-67.
Cooper, P. R., Lamb, R., Day, N. D., Branigan, P. J., Kajekar, R., San Mateo, L., et al. (2009). TLR3 activation stimulates cytokine secretion without altering agonist-induced human small airway contraction or relaxation. Am. J. Physiol.—Lung Cell. Mol. Physiol. 297: L530-L537.
Curtis, M. J., Bond, R. A., Spina, D., Ahluwalia, A., Alexander, S. P. A., Giembycz, M. A., et al. (2015). Experimental design and analysis and their reporting: new guidance for publication in BJP. 3461-3471.
Desensitization, G. R. K., Kong, K. C., Gandhi, U., Martin, T. J., Anz, C. B., Yan, H., et al. (2008).
Endogenous G s -Coupled Receptors in Smooth Muscle Exhibit Differential. Group 9279-9288.
Deshpande, D. A., Wang, W. C. H., Mcllmoyle, E. L., Robinett, K. S., Schillinger, R. M., An, S. S., et al. (2010). Bitter taste receptors on airway smooth muscle bronchodilate by localized calcium signaling and reverse obstruction. Nat Med 16: 1299-1304.
Deshpande, D. A., Yan, H., Kong, K.-C., Tiegs, B. C., Morgan, S. J., Pera, T., et al. (2014). Exploiting functional domains of GRK2/3 to alter the competitive balance of pro- and anticontractile signaling in airway smooth muscle. FASEB J. 28: 956-965.
Fabry, B., Maksym, G. N., Butler, J. P., Glogauer, M., Navajas, D., and Fredberg, J. J. (2001). Scaling the Microrheology of Living Cells. Phys. Rev. Lett. 87: 148102.
Fisher, J. T., Vincent, S. G., Gomeza, J., Yamada, M., and Wess, J. (2004). Loss of vagally mediated bradycardia and bronchoconstriction in mice lacking M2 or M3 muscarinic acetylcholine receptors. FASEB J. 18: 711-713.
Goncharova, E. a, Ammit, A. J., Irani, C., Carroll, R. G., Eszterhas, A. J., Panettieri, R. a, et al. (2002). PI3K is required for proliferation and migration of human pulmonary vascular smooth muscle cells. Am. J. Physiol. Lung Cell. Mol. Physiol. 283: L354-L363.
Himes, B. E., Koziol-White, C., Johnson, M., Nikolos, C., Jester, W., Klanderman, B., et al. (2015). Vitamin D modulates expression of the airway smooth muscle transcriptome in fatal asthma. PLoS One 10.
Jude, J. A., Tirumurugaan, K. G., Kang, B. N., Panettieri, R. A., Walseth, T. F., and Kannan, M. S. (2012). Regulation of CD38 expression in human airway smooth muscle cells: Role of class I phosphatidylinositol 3 kinases. Am. J. Respir. Cell Mol. Biol. 47: 427-435.
Koziol-White, C. J., and Panettieri, R. A. (2011). Airway smooth muscle and immunomodulation in acute exacerbations of airway disease. Immunol. Rev. 242: 178-185.
Koziol-White, C. J., Yoo, E. J., Cao, G., Zhang, J., Papanikolaou, E., Pushkarsky, I., et al. (2016). Inhibition of phosphoinositide 3-kinase (PI3K) promotes bronchodilation of human small airways in a Rho kinase-dependent manner. Br. J. Pharmacol. (accepted).
Krymskaya, V. P., Orsini, M. J., Eszterhas, A. J., Brodbeck, K. C., Benovic, J. L., Panettieri, R. A., et al. (2000). Mechanisms of proliferation synergy by receptor tyrosine kinase and G protein-coupled receptor activation in human airway smooth muscle. Am J Respir Cell Mol Biol 23: 546-554.
Lee, S. J., Lee, W. H., Ki, S. H., Kim, Y. M., Lee, S. J., Lee, C. H., et al. (2009). Gα13 regulates methacholine-induced contraction of bronchial smooth muscle via phosphorylation of MLC20. Biochem. Pharmacol. 77: 1497-1505.
Leopoldt, D., Hanck, T., Exner, T., Maier, U., Wetzker, R., and Nürnberg, B. (1998). Gbg stimulates phosphoinositide 3-kinase-g by direct interaction with two domains of the catalytic p110 subunit. J. Biol. Chem. 273: 7024-7029.
MURTHY, K. S., ZHOU, H., GRIDER, J. R., BRAUTIGAN, D. L., ETO, M., and MAKHLOUF, G. M. (2003). Differential signalling by muscarinic receptors in smooth muscle: m2-mediated inactivation of myosin light chain kinase via Gi3, Cdc42/Rac1 and p21-activated kinase 1 pathway, and m3-mediated MLC20 (20 kDa regulatory light chain of myosin II) phosphorylat. Biochem. J. 374: 145.
Nieuwstadt, R. A. van, Henricks, P. A., Hajer, R., Meer van Roomen, W. A. van der, Breukink, H. J., and Nijkamp, F. P. (1997). Characterization of muscarinic receptors in equine tracheal smooth muscle in vitro.
Panettieri, R. A. (2016). Neutrophilic and Pauci-immune Phenotypes in Severe Asthma. Immunol. Allergy Clin. North Am. 36: 569-579.
Panettieri, R. A., Murray, R. K., DePalo, L. R., Yadvish, P. A., and Kotlikoff, M. I. (1989a). A human airway smooth muscle cell line that retains physiological responsiveness. Am J Physiol 256: C329-35.
Panettieri, R. A., Murray, R. K., DePalo, L. R., Yadvish, P. A., and Kotlikoff, M. I. (1989b). A human airway smooth muscle cell line that retains physiological responsiveness. Am J Physiol 256: C329-35.
Penn, R. B., and Benovic, J. L. (2008). Regulation of heterotrimeric G protein signaling in airway smooth muscle. Proc. Am. Thorac. Soc. 5: 47-57.
Pera, T., Hegde, A., Deshpande, D. A., Morgan, S. J., Tiegs, B. C., Theriot, B. S., et al. (2015). Specificity of arrestin subtypes in regulating airway smooth muscle G protein-coupled receptor signaling and function. FASEB J. Off. Publ. Fed. Am. Soc. Exp. Biol. 29: 4227-4235.
Pera, T., and Penn, R. B. (2016). Bronchoprotection and bronchorelaxation in asthma: New targets, and new ways to target the old ones. Pharmacol. Ther. 164: 82-96.
Riobo, N. A., and Manning, D. R. (2005). Receptors coupled to heterotrimeric G proteins of the G12 family. Trends Pharmacol. Sci. 26: 146-154.
Roffel, A. F., Elzinga, C. R. S., Amsterdam, R. G. M. Van, Zeeuw, R. A. De, and Zaagsma, J. (1988). Muscarinic M2 receptors in bovine tracheal smooth muscle: discrepancies between binding and function. Eur. J. Pharmacol. 153: 73-82.
Roffel, A. F., Elzinga, C. R. S., and Zaagsma, J. (1993). Cholinergic contraction of the guinea pig lung strip is mediated by muscarinic M2-like receptors. Eur. J. Pharmacol. 250: 267-279.
Roffel, a F., Elzinga, C. R., and Zaagsma, J. (1990). Muscarinic M3 receptors mediate contraction of human central and peripheral airway smooth muscle. Pulm. Pharmacol. 3: 47-51.
Rümenapp, U., Asmus, M., Schablowski, H., Woznicki, M., Han, L., Jakobs, K. H., et al. (2001). The M3 muscarinic acetylcholine receptor expressed in HEK-293 cells signals to phospholipase D via G12 but not Gq-type G proteins. Regulators of G proteins as tools to dissect pertussis toxin-resistant G proteins in receptor-effector coupling. J. Biol. Chem. 276: 2474-2479.
Shaifta, Y., Irechukwu, N., Prieto-Lloret, J., Mackay, C. E., Marchon, K. A., Ward, J. P. T., et al. (2015). Divergent modulation of Rho-kinase and Ca2+ influx pathways by Src family kinases and focal adhesion kinase in airway smooth muscle. Br. J. Pharmacol. 172: 5265-5280.
Shang, X., Marchioni, F., Sipes, N., Evelyn, C. R., Jerabek-Willemsen, M., Duhr, S., et al. (2012). Rational design of small molecule inhibitors targeting RhoA subfamily Rho GTPases. Chem. Biol. 19: 699-710.
Siehler, S. (2009). Regulation of RhoGEF proteins by G 12/13-coupled receptors. Br. J. Pharmacol. 158: 41-49.
Struckmann, N., Schwering, S., Wiegand, S., Gschnell, A., Yamada, M., Kummer, W., et al. (2003). Role of muscarinic receptor subtypes in the constriction of peripheral airways: studies on receptor-deficient mice. Mol. Pharmacol. 64: 1444-1451.
Tseng, P., Pushkarsky, I., and Carlo, D. Di (2014). Metallization and biopatterning on ultra-flexible substrates via dextran sacrificial layers. PLoS One 9.
Wang, Z. (2016). Transactivation of epidermal growth factor receptor by g protein-coupled receptors: Recent progress, challenges and future research. Int. J. Mol. Sci. 17.
Wells, C. D., Liu, M. Y., Jackson, M., Gutowski, S., Sternweis, P. M., Rothstein, J. D., et al. (2002). Mechanisms for reversible regulation between G13 and Rho exchange factors. J. Biol. Chem. 277: 1174-1181.
Worzfeld, T., Wettschureck, N., and Offermanns, S. (2008). G12/G13-mediated signalling in mammalian physiology and disease. Trends Pharmacol. Sci. 29: 582-589.
Yang, H. W., Shin, M. G., Lee, S., Kim, J. R., Park, W. S., Cho, K. H., et al. (2012). Cooperative Activation of PI3K by Ras and Rho Family Small GTPases. Mol. Cell 47: 281-290.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1.-13. (canceled)

14. A method of treating bronchoconstriction in a subject in need thereof, the method comprising administering to the subject a therapeutically effective amount of a Gα₁₂ inhibitor.

15. The method of claim 14, wherein the Gα₁₂ inhibitor is a nucleic acid, a peptide, an antibody, a peptidomimetic or a small molecule.

16. The method of claim 15, wherein the Gα₁₂ inhibitor is a short interfering ribonucleic acid (siRNA).

17. The method of claim 15, wherein the Gα₁₂ inhibitor is a polypeptide comprising a regulator of G-protein signaling (RGS) domain.

18.-19. (canceled)

20. A method of treating bronchoconstriction in a subject in need thereof, comprising administering to the subject a therapeutically effective amount of a ras homolog gene family, member A (RhoA) inhibitor.

21.-23. (canceled)

24. The method of claim 14, wherein the subject has a disease characterized by bronchoconstriction.

25. The method of claim 24, wherein the disease is asthma, chronic obstructive pulmonary disease (COPD), chronic bronchitis, bronchiectasis or cystic fibrosis.

26. The method of claim 14, wherein the subject has airway hyperresponsiveness.

27. The method of claim 14, wherein the subject is a human.

28. The method of claim 14, wherein the inhibitor is administered by inhalation.

29. The method of claim 14, wherein the inhibitor is administered orally.

30. The method of claim 14, further comprising administering to the subject a therapeutically effective amount of one or more additional agents.

31. The method of claim 30, wherein the one or more additional agents is selected from a beta-adrenergic agonist, an anti-inflammatory agent or an agent that inhibits activation of the phosphoinositide 3-kinase (PI3K)/rho kinase (ROCK) axis.

32. A pharmaceutical composition comprising a Gα₁₂ inhibitor, a RhoA inhibitor, or a combination thereof; and a pharmaceutically acceptable carrier.

33.-42. (canceled)

43. The pharmaceutical composition of claim 32, wherein the therapeutically effective amount of the inhibitor is a therapeutically effective amount to treat or prevent airway hyperresponsivness.

44. The pharmaceutical composition of claim 32, further comprising one or more additional agents.

45.-46. (canceled)

47. A method of identifying an agent that inhibits contraction or promotes relaxation of an airway smooth muscle cell, comprising:
contacting an airway smooth muscle cell with a contractile agent and a candidate agent that inhibits contraction or promotes relaxation of an airway smooth muscle cell; and
measuring activation of the phosphoinositide 3-kinase (PI3K)/rho kinase (ROCK) axis in the airway smooth muscle cell,
wherein a reduction in activation of the PI3K/ROCK axis in an airway smooth muscle cell that has been contacted with the candidate agent compared to a control indicates that the candidate agent inhibits contraction or promotes relaxation of an airway smooth muscle cell.

48.-50. (canceled)

51. The method of claim 14, further comprising administering to the subject a therapeutically effective amount of a RhoA inhibitor.

52. The method of claim 51, wherein the RhoA inhibitor is a nucleic acid, a peptide, an antibody, a peptidomimetic or a small molecule.

53. The method of claim 51, wherein the RhoA inhibitor is rhosin.
